(19) United States
(12) Patent Application Publication
(10) Pub. No.: US 2018/0243343 A1
(43) Pub. Date: Aug. 30, 2018

(54) METHODS AND COMPOSITIONS FOR THE TREATMENT OR PREVENTION OF PARKINSON'S DISEASE

(71) Applicant: THE MCLEAN HOSPITAL CORPORATION, Belmont, MA (US)

(72) Inventor: Ole Isacson, Cambridge, MA (US)

(73) Assignee: THE MCLEAN HOSPITAL CORPORATION, Belmont, MA (US)

(21) Appl. No.: 15/553,318

(22) PCT Filed: Feb. 26, 2016
(86) PCT No.: PCT/US16/19798

§ 371 Date: Aug. 24, 2017

Related U.S. Application Data
(60) Provisional application No. 62/121,333, filed on Feb. 26, 2015.

(51) Int. Cl. A61K 35/30; A61K 38/18; A61P 25/16; C12N 5/0793

(52) U.S. Cl. CPC A61K 35/30; A61K 38/185; A61P 25/16; C12N 2501/10; C12N 2506/45; C12N 2501/999; C12N 2501/40; C12N 5/0619

(57) ABSTRACT

Provided are methods and compositions for the treatment and/or prevention of Parkinson's disease in humans. Specifically, the compositions comprising: (a) a population of cells comprising at least 3% dopaminergic neurons, wherein the dopaminergic neurons express FOXA2, Beta-tubulin, and tyrosine hydroxylase (TH), (b) a neurotrophic factor, and (c) a pharmaceutically acceptable carrier; and methods for the treatment and/or prevention of Parkinson's disease comprising administering or transplanting a composition comprising dopaminergic neurons derived from the patient's own cells.

### CROSS-REFERENCE TO RELATED APPLICATION

This Application claims benefit under 35 U.S.C. § 119(e) of the U.S. Provisional Application No. 62/121,333 filed Feb. 26, 2015, the contents of which are incorporated herein by reference in their entirety.

### FIELD OF THE DISCLOSURE

The present disclosure relates to cell compositions and their use in the treatment and/or prevention of Parkinson's Disease.

### BACKGROUND

Neurodegenerative disorders such as Parkinson's, Alzheimer's, and Huntington's disease are becoming ever more prominent in our society. Parkinson's disease (PD) is a progressive neurodegenerative disease characterized clinically by bradykinesia, rigidity, and resting tremor. The motor abnormalities are associated with a specific loss of dopaminergic neurons in the substantia nigra pars compacta (SN) and depletion of striatal dopamine (DA) levels. While the loss of striatal DA correlates with the severity of clinical disability, clinical manifestations of PD are not apparent until about 80-85% of SN neurons have degenerated and striatal DA levels are depleted by about 60-80%.

DA neurons in the ventral midbrain consist of two main groups: the A9 group in the SN, and the A10 group in the medial and ventral tegmentum. Each of these cell groups project to different anatomical structures and is involved in distinct functions. A9 cells mainly project to the dorsolateral striatum, and are involved in the control of motor functions, whereas A10 cells provide connections to the ventromedial striatum, limbic and cortical regions, and are involved in reward and emotional behavior. In addition to the distinct axonal projections and differences in synaptic connectivity, these groups of DA cells exhibit differences in neurochemistry and electrophysiological properties, illustrating functional differences despite similar neurotransmitter identity. These differences in A9 and A10 cells are also reflected in their specific responses to neurodegeneration in PD. Postmortem analyses in human PD brains demonstrate a selective cell loss of the A9 group with a survival rate of about 10% whereas the A10 group is largely spared with a survival rate of about 60%. This indicates that A9 cells are more vulnerable to intrinsic and/or extrinsic factors causing degeneration in PD. In addition, three regional gradients of neurodegeneration in the dorso-ventral/rostro-caudal/medio-lateral axis have been reported in PD. Caudally and laterally located ventral DA cells within A9 subgroups are the most vulnerable cells in PD. In contrast, the medial and rostral part of DA cell subgroups within A10 cells (i.e. rostral linear nucleus, RLi) are the least affected (5-25% cell loss).

Cell transplantation therapies have been used to treat neurodegenerative disease, including Parkinson's disease, with moderate success (e.g., Bjorklund et al., Nat. Neurosci., 3:537-544, 2000). However, wide-spread application of cell-based therapies will depend upon the availability of sufficient amounts of neuronal cells.

### SUMMARY

Provided herein are methods and compositions for the treatment and/or prevention of Parkinson's disease by administering or transplanting a composition comprising dopaminergic neurons derived from the patient's own cells.

Accordingly, provided herein in one aspect is a composition for the treatment and/or prevention of Parkinson's Disease, the composition comprising: (a) a population of cells comprising at least 3% dopaminergic neurons, wherein the dopaminergic neurons express FOXA2, β-tubulin, and tyrosine hydroxylase (TH), (b) a neurotrophic factor, and (c) a pharmaceutically acceptable carrier.

In one embodiment of this aspect and all other aspects provided herein, the neurotrophic factor is selected from the group consisting of: glial cell line-derived neurotrophic factor (GDNF), neurturin (NRTN), artemin (ARTN), and persephin (PSPN).

In another embodiment of this aspect and all other aspects provided herein, the neurotrophic factor is GDNF.

In another embodiment of this aspect and all other aspects provided herein, the population of cells comprises at least 20% dopaminergic neurons.

In another embodiment of this aspect and all other aspects provided herein, the population of cells comprises between 4-40% dopaminergic neurons.

In another embodiment of this aspect and all other aspects provided herein, the population of cells is free of teratoma-forming cells.

In another embodiment of this aspect and all other aspects provided herein, the teratoma-forming cells express TRA60 and/or SSEA.

In another embodiment of this aspect and all other aspects provided herein, the pharmaceutically acceptable carrier is artificial cerebrospinal fluid.

In another embodiment of this aspect and all other aspects provided herein, the dopaminergic neurons are post-mitotic cells.

In another embodiment of this aspect and all other aspects provided herein, the volume of the composition is less than 1 mL.

In another embodiment of this aspect and all other aspects provided herein, the population of cells comprises at least 10,000 dopaminergic neurons.

In another embodiment of this aspect and all other aspects provided herein, the composition is xeno-free.

In another embodiment of this aspect and all other aspects provided herein, the dopaminergic neurons are footprint free.

In another embodiment of this aspect and all other aspects provided herein, the dopaminergic neurons further express G-protein inwardly-rectifying potassium channel (GIRK)-2.

Another aspect provided herein relates to a method for treating and/or preventing Parkinson's disease, the method comprising: administering a composition as described herein into the caudate, putamen, nucleus accumbens, or subthalamic nucleus of a subject in need thereof, thereby treating and/or preventing Parkinson's disease.

In one embodiment of this aspect and all other aspects provided herein, the composition is administered to the putamen.

In another embodiment of this aspect and all other aspects provided herein, the composition is administered bilaterally.

In another embodiment of this aspect and all other aspects provided herein, the composition is administered using stereotactic injection.

In another embodiment of this aspect and all other aspects provided herein, the subject is diagnosed as having Parkinson's disease and/or is monitored following transplantation by positron emission tomography (PET) scanning.

In another embodiment of this aspect and all other aspects provided herein, the subject is diagnosed or monitored for successful transplantation using the Unified Parkinson's Disease Rating Scale (UPRDS).

In another embodiment of this aspect and all other aspects provided herein, the subject is a human.

In another embodiment of this aspect and all other aspects provided herein, the method is repeated.

In another embodiment of this aspect and all other aspects provided herein, the method is repeated until the subject is substantially free of Parkinson's disease symptoms.

In another embodiment of this aspect and all other aspects provided herein, wherein the method restores the number of functional neurons to at least 60% of the functional dopaminergic synapses normally observed in the putamen.

In another embodiment of this aspect and all other aspects provided herein, the method reduces the dose of L-DOPA required by the subject to achieve adequate clinical control of symptoms.

Another aspect provided herein relates to a method for reducing the dose of L-DOPA required by a Parkinson's patient to achieve adequate clinical control of symptoms, the method comprising: (a) administering a composition as described herein into the caudate, putamen, nucleus accumbens, or subthalamic nucleus of a subject receiving L-DOPA to control Parkinson's symptoms, (b) monitoring the subject for engraftment of dopaminergic neurons and/or for side effects associated with L-DOPA, and (c) reducing or eliminating the dose of L-DOPA as required to reduce L-DOPA associated side effects while maintaining adequate clinical control of Parkinson's symptoms.

Another aspect provided herein relates to a method of treating and/or preventing Parkinson's disease, the method comprising the steps outlined in FIG. 10.

Another aspect provided herein relates to a method comprising: (a) reprogramming somatic cells obtained from a subject into iPS cells, (b) contacting the iPS cells with retinoic acid, human SHH and FGF8A to promote differentiation of the iPS cells into dopaminergic neurons, wherein the dopaminergic neurons express FOXA2, β-tubulin and tyrosine hydroxylase, and (c) depleting the population of cells of step (b) of teratoma-forming cells.

In one embodiment of this aspect and all other aspects provided herein, the transplant composition comprises at least 4% dopaminergic neurons that express FOXA2, β-tubulin and tyrosine hydroxylase.

In another embodiment of this aspect and all other aspects provided herein, the dopaminergic neurons further express GIRK2.

In another embodiment of this aspect and all other aspects provided herein, the teratoma-forming cells are depleted from the population of cells of step (b) by removing cells expressing TRA160 and/or SSEA.

In another embodiment of this aspect and all other aspects provided herein, the somatic cells are peripheral blood mononuclear cells (PBMCs) or fibroblasts.

In another embodiment of this aspect and all other aspects provided herein, the method further comprises a step of freezing and thawing the population of cells of step (b) or step (c).

In another embodiment of this aspect and all other aspects provided herein, the method further comprises admixing the depleted culture of step (c) with a pharmaceutically acceptable carrier.

Another aspect described herein relates to the use of a composition as described herein for the treatment and/or prevention of Parkinson's disease.

### BRIEF DESCRIPTION OF THE FIGURES

FIG. 1 is a schematic representation of exemplary methods for treating Parkinson's Disease.

FIGS. 2A-2E Functional Improvement of PD Motor Symptoms, Increased Dopamine Reuptake, and Reinnervation of the Transplanted Putamen after Autologous Transplantation of CM iPSC-Derived Dopamine Neurons. FIG. 2A, Differentiated CM-iPSCs were transplanted unilaterally into the putamen of three CMs with stable, bilateral parkinsonism (MF25-04, MF66-02, MF27-04). The animals were followed for 1-2 years after transplantation. From 6 months after transplantation, functional improvement was observed in MF25-04, as determined by a sustained increase in global daytime (6 a.m. to 6 p.m.) activity. The non-transplanted control group and non-surviving transplant group represent the average data of n=4 and n=3 animals, respectively, and error bars show the SEM. FIG. 2B, Fine-motor skills in MF25-04 were assessed using a computerized reaching task MAP. At 2 years after transplantation, MAP performance in the left upper limb was significantly improved compared with pre-transplantation values (p<0.05, one-way ANOVA followed by Tukey's multiple comparison test). No change in performance was observed in the right upper limb. Data shown represent averages of five repeated tests (baseline), two repeated tests (1 year after transplantation), and three repeated tests (2 years after transplantation). Error bars represent the SEM.

FIG. 2C, Functional analysis of dopamine reuptake in MF25-04 was measured by PET neuroimaging for 11 C-CFT, a marker of the DAT. Increased 11 C-CFT binding was observed in the transplanted putamen at 2 years after transplantation. White arrows indicate areas of hyperintense CFT PET signal.

FIG. 2D, Low-power photomicrograph of DAT immunostaining in the transplanted (right, R) and non-transplanted (left, L) putamen in MF25-04 shows reinnervation of the transplanted side. Deposits of grafted dopamine neurons are indicated with boxes (g). IC, internal capsule; LGP, lateral globus pallidus; LV, lateral ventricle; 3V, third ventricle; cc, corpus callosum. FIG. 2E, Grafted dopamine neurons were also labeled using tyrosine hydroxylase (TH). The boxed area is shown at higher magnification in the right. Robust survival of dopamine neurons with outgrowth integration into the host putamen was observed.

FIGS. 3A-3H Phenotypes of Engrafted CM iPSC-Derived Neurons at 2 Years after Autologous Transplantation Postmortem immunohistochemical analysis was used to further characterize the graft in MF25-04. FIG. 3A, Immunofluorescence staining confirmed that transplanted dopamine neurons were co-labeled for FOXA2, TH, and bIII tubulin at 2 years after transplantation. FIG. 3B, Labeling for DAT, TH, and TOM20 (a mitochondrial outer membrane protein) showed a punctate expression of DAT along the fibers of transplanted dopamine neurons and a typical localization of mitochondria throughout the cell soma and neurites. FIG. 3C, Immunofluorescence staining for 5-HT demonstrated the presence and localization of serotonergic neurons within the graft. FIG. 3D, Labeling for DARPP-32, a marker of striatal GABAergic medium spiny neurons, shows robust labeling in the host putamen and occasional DARPP-32-ir fibers at the graft-host border. FIGS. 3E & 3F, Ki-67 was used to determine whether proliferating cells were present in the CM-iPSC-derived neural cell graft from MF25-04. No Ki-67-immunoreactive cells were observed in the graft (FIG. 3E). As a positive control, several Ki-67-immunoreactive proliferating cells were observed in the hippocampus of the same animal (FIG. 3F) (identified with arrows). FIGS. 3G & 3H, Histological analysis of microglia using Iba1 in the host putamen (FIG. 3G) and graft (FIG. 3H) shows typical resting microglia.

FIGS. 4A-4E Generation of dopamine neurons from CM iPSCs. FIG. 4A, CM iPSCs derived from MF25-04 were differentiated toward midbrain-like dopamine neurons using an established differentiation protocol that supports floor plate regionalization of cells into FOXA2 dopamine neurons (Cooper et al., 2010). FIGS. 4B-4E, At day in vitro (DIV) 47, immunocytochemistry was used to determine the percentage of neurons labeled for βIII-tubulin, tyrosine hydroxylase (TH) and FOXA2 prior to in vivo transplantation of differentiated neurons on DIV49.

FIGS. 5A-5I Dopaminergic reinnervation of the host putamen following autologous transplantation of CM iPSC-derived dopamine neurons, and Iba1-ir microglia in CM iPSC-derived grafts in MF27-04 and MF66-02, and in a non-surviving transplant. FIGS. 5A-5C, Immunofluorescence staining for TH showed extensive dopaminergic innervation of the host putamen in the grafted putamen of MF25-04, following autologous transplantation of CM iPSC-derived neural cells, and comparatively less innervation in MF27-04 (FIG. 5B) and MF66-02 (FIG. 5C). Insets show TH labeling in a comparative region of the putamen on the contralateral (untransplanted) side. Lateral ventricle (LV). FIGS. 5D-5F, Immunofluorescence staining for FOXA2 and TH indicated that a greater number of transplanted dopamine neurons were co-labeled for FOXA2 and TH in the graft of MF25-04 (FIG. 5D), compared to the grafts in MF27-04 (FIG. 5E) and MF66-02 (FIG. 5F). Examples of FOXA2/TH co-labeled neurons are identified using asterisks. FIGS. 5G-5I, Histological analysis of microglia using Iba1 in the putamen, shows a mildly increased intensity of Iba1 staining in the needle tract (nt) of a non-surviving transplant (FIG. 5G), and in the area of the graft in CM iPSC-derived grafts from MF27-04 (FIG. 5H), and MF66-02 (FIG. 5I).

FIG. 6 Synopsis of cell differentiation method and clinical score alterations of animals included in study. CM-iPSCs and parthenogenetic ES cells (Cyno-1) were differentiated according to Cooper et al. (Cooper et al., 2010), Sundberg et al. (Sundberg et al., 2013), or Sanchez-Pernaute et al. (Sanchez-Pernaute et al., 2005). Duration of study indicates the time of survival post-transplantation. The Parkinsonian Rating Scale (PRS) was modified from the motor subscale of the Unified Parkinson's Disease Rating Scale (UPDRS) and developed for macaques (Imbert et al., 2000). The PRS ranged from 0-24, with 24 being most severe Global motor activity scores were collected using activity monitors (AW64 Actiwatch, Philips Electronics) (Mann et al., 2005).

FIGS. 7A-7C Xeno-free differentiation of iPSCs toward midbrain DA neurons. A feeder-free and xeno-free version of a previously published midbrain dopaminergic differentiation protocol (Cooper et al 2010) was developed by replacing MS5 stromal cells by a humanized substrate, use of small molecules instead of recombinant proteins and the use of xeno-free media. FIG. 7A, Midbrain DA neurons, as identified by co-staining of TH and FoxA2 are generated from the xeno-free protocol. The percentage of TH/FoxA2+ neurons at DIV 49 is equivalent to what is generated using mouse feeder cells. FIGS. 7A & 7B, The Ki67 positive cells are mainly nestin positive, indicating dividing cells come from a population of neural precursor cells. The percentages of BIII tubulin positive cells are also equivalent in the three different protocols. Approximately 25-35% of the cultures are BIII tubulin positive. FIG. 7C, Midbrain DA neurons are present 10 weeks after grafting into striatum in rats (stains include (i) human NCAM, (ii) TH and (iii) FoxA2) Scale bar: 20 μM.

FIGS. 8A-8C, Midbrain DA neurons generated under xeno-free and feeder free conditions survive long-term after xenogeneic implantation and result in behavioral recovery of hemi-parkinsonian rats. Cells were implanted at DIV49 to the striatum of 6-OHDA lesioned rats. FIG. 8A, Amphetamine-induced rotations significantly declined over time and were significantly less compared to lesioned control rats 20 weeks after transplantation. FIG. 8B, Contralateral paw use (assessed in the cylinder test) was significantly improved 24 weeks post-transplantation. FIG. 8C, TH positive neurons were observed throughout the graft 24 weeks after transplantation.

FIGS. 9A-9C The mature xenogeneic grafts do not contain proliferating cells. Rats were given BrdU injections (50 mg/kg/day) daily for 12 days. FIG. 9A, Proliferating cells in the subventricular zone were efficiently labeled (positive control). FIG. 9B, 7 weeks after transplantation proliferating cells could still be observed in the graft. FIG. 9C, By 6 months there were no graft derived proliferating cells observed.

FIG. 10 An exemplary protocol for an autologous transplantation of iPS-derived dopaminergic neurons into the putamen. Exemplary methods for transplantation as shown in FIG. 10 include cell numbers, number of deposits, number of deposition sites or tracts, flow rate for administration, cell concentration etc. It is important to note that the protocol in FIG. 10 is modified to shorten the original protocol described by Cooper et al. from 49 days to 28 days. The methods outlined in FIG. 10 are mere examples and can be further optimized by one of skill in the art for optimal treatment and/or prevention of Parkinson's disease.

### DETAILED DESCRIPTION

The methods and compositions provided herein are based, in part, on the development and optimization of compositions and methods for the treatment of Parkinson's disease in humans. The methods and compositions utilize a patient's somatic cells to generate iPS cells for differentiation into dopaminergic neurons.

### Definitions

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein and includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. In another embodiment, the subject is a domesticated animal including companion animals (e.g., dogs, cats, rats, guinea pigs, hamsters etc.).

As used herein, the term “induced pluripotent stem cell” (iPS cell) refers to pluripotent cells derived from somatic cells (e.g., a skin cell, a fibroblast, a blood cell, a peripheral blood mononuclear cell (PBMC), etc) through the induced expression of one or more transcription factors. The iPS cells are capable of self-renewal and subsequent differentiation into more than one specialized cell type or cell lineage under appropriate growth conditions either in vitro or in vivo. In one specific embodiment, iPS cells are derived from fibroblasts by the overexpression of Oct4, Sox2, c-Myc and Klf4 according to the methods described in Takahashi et al. (Cell, 126:663-676, 2006), for example. Other methods for producing iPS cells are described, for example, in Takahashi et al. (Cell, 131:861-872, 2007) and Nakagawa et al. (Nat. Biotechnol., 26:101-106, 2008). When human iPS cells are used to derive dopaminergic neurons for the administration to a human, it is preferred to produce the iPS cells using a method that does not leave a “footprint” in the cells. That is, iPS cells used for differentiation and treatment of a human should be produced using induced expression of one or more transcription factors that does not encompass the use of a vector that inserts a nucleic acid sequence into the genome. Thus, in one embodiment, iPS cells for use in methods for treating humans are generated by induced expression of at least one transcription factor (e.g., at least 2, at least 3, at least 4 or more transcription factors) by contacting the somatic cell with at least one non-inserting vector, plasmids, protein, modified RNA or small molecule. Upon generation of the iPS cells, the cells can be grown and passaged in culture to dilute out non-inserting vectors or plasmids such that the iPS cells do not comprise an exogenous vector and are considered “footprint free.”

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof). In one embodiment, the term “treating” can refer to cure of Parkinson's disease as assessed using the Unified Parkinson's disease rating scale.

As used herein, the term “xeno-free,” when referring to cell media components, means that the media components are produced using proteins, growth factors, and other components that are the same species as the subject to be treated. For example, where humans are to be treated, “xeno-free” refers to cell media and growth components that are entirely free from non-human components and production methods. That is, xeno-free components include protein and/or nucleic acid sequences specific to humans and are produced either in vitro or in vivo without contact with a component derived from another species (e.g., mouse, rats, horses, dogs, guinea pigs, etc) or in which any non-human components have been removed and are below the level of standard detection methods.

As used herein, the term “footprint free” refers to a cell or composition that is produced using transient overexpression of reprogramming or differentiation factors that do not cause integration of a nucleic acid into the genome of the cell or permanent changes of the genome of the cell. That is, methods of reprogramming or differentiation to produce footprint free cells comprises administration of e.g., proteins, modified RNAs, non-integrating vectors (e.g., adenoviral vectors), plasmids, or small molecules to effect reprogramming but does not encompass the use of vectors that permit integration of a nucleic acid into the genome of the cell (e.g., retroviruses, such as lentivirus).

As used herein, the term “substantially free from Parkinson's symptoms” refers to a reduction in clinical symptoms of Parkinson's such that the subject does not need supportive treatment, such as L-DOPA, to control or alleviate Parkinson's symptoms. A skilled clinician can easily determine whether a subject is “substantially free from Parkinson's disease symptoms” from assessing a subject based on the Unified Parkinson's Disease Rating Scale (UPRDS).

As used herein, the term “adequate clinical control of symptoms” refers to a level of symptoms that does not warrant a further increase in L-DOPA or another supportive treatment of Parkinson's disease as deemed by a skilled clinician working in concert with the patient. While “adequate clinical control” may not mean a complete reduction in symptoms, the term refers to a level of symptom control where a further increase in the dose of the treatment merely increases the side effects of the treatment without providing further symptom control. A skilled clinician can work with a patient to determine, on an individual basis, what an adequate or acceptable level of clinical control of symptoms is for that patient.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”,“increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “pharmaceutically acceptable” can refer to compounds and compositions which can be administered to a subject (e.g., a mammal or a human) without undue toxicity.

As used herein, the term “pharmaceutically acceptable carrier” can include any material or substance that, when combined with an active ingredient allows the ingredient to retain biological activity and is non-reactive with the subject's immune system. Examples include, but are not limited to, any of the standard pharmaceutical carriers such as a phosphate buffered saline solution, water, emulsions such as oil/water emulsion, artificial cerebral spinal fluid and various types of wetting agents. The term “pharmaceutically acceptable carriers” excludes tissue culture media.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

It should be understood that this invention is not limited to the particular methodologies, protocols, and reagents, etc., described herein and as such can vary therefrom. The terminology used herein is for the purpose of describing particular embodiments only, and is not intended to limit the scope of the present invention, which is defined solely by the claims.

### Cell Therapy in Parkinson's Disease

A major pathological hallmark of Parkinson's disease (PD) is the progressive loss of dopamine (DA) neurons in the substantia nigra pars compacta (SNc), which subsequently causes a dysregulation of striatal neurotransmission and development of motor symptoms of PD (bradykinesia, ridigity, tremor). If possible, it would be reasonable to initiate neuroprotective strategies at preclinical stages of disease to prevent further neuronal degeneration. However, in reality most patients remain free of clinical motor symptoms until the PD pathology has already reached an advanced stage with most (˜60%) of the selectively vulnerable dopamine neurons dysfunctional or dead, and with a consequent depletion of roughly 70% of striatal dopamine. For this reason, and since any retardation of degeneration is unlikely to be absolute, it is rational to develop cell replacement for the lost neurons. Such “live cell” replacement therapies are conceptually different from classical pharmacology. The current mainstay treatment for PD is based on a pharmacological approach using levodopa (L-dopa) or dopaminergic agonists that elevate dopamine levels or stimulate dopamine receptors. Although this treatment can be effective for many years, its long-term and chronic use can result in the development of “motor complications”, including wearing-off, on-off fluctuations and abnormal movements termed L-dopa-induced dyskinesias. L-dopa crosses the blood-brain-barrier where it is converted to dopamine by dopa-decarboxylase containing cells; these include the remaining striatal dopaminergic terminals themselves, and also non-dopaminergic cells including cells in the blood-brain-barrier wall and serotonin neurons. Conversion of L-dopa to dopamine in non-dopaminergic cells following oral (non-continuous) administration of L-dopa, results in a pulsatile, non-physiological release of dopamine, which may act on supersensitive dopamine receptors in the striatum and contribute to the development of dyskinesias.

Cell based therapy approaches in PD aim to replace nigrostriatal dopamine terminals lost by the disease process, with fetal or stem-cell derived dopamine neurons placed directly into the caudate-putamen and substantia nigra. Cell replacement therapy with midbrain dopamine neurons in PD addresses both the motor symptoms of PD, as well as L-dopa-induced dyskinesias. In the most successful cases, the requirement for L-dopa medication has been negated or substantially reduced. When new midbrain-like dopamine neurons are engrafted into the normal target regions of nigrostriatal dopamine neurons, they establish synapses with mature host striatal neurons and provide physiologically appropriate dopamine release and synaptic feedback control in the host brain (Vinuela et al., 2008; Zetterstrom et al., 1986). Replacement of dopamine terminals in this manner may be far more effective in ameliorating the motor symptoms of PD than a pump-like pharmacological delivery of dopamine into the striatum that lacks physiological release and reuptake mechanisms.

Long-term remarkable and neurologically clear benefits (approximately 50-60% reduction in Unified Parkinson's Disease Rating Scale (UPDRS) scores off DA drug therapy) to PD patients following fetal dopamine neuron transplantation has been reported for over 18 years (Hallett et al., 2014; Kefalopoulou et al., 2014; Barker et al., 2013; Mendez et al., 2005; Politis et al., 2010), and this outcompetes any current treatment for PD. Moreover, implanted fetal dopamine neurons can prevent progressive worsening of PD motor scores over at least 14 years ((Kefalopoulou et al., 2014). These clinical benefits are associated with evidence of physiological changes using PET and functional MRI neuroimaging.

Previous data from the inventors also shows that transplanted fetal ventral midbrain dopamine neurons remain healthy long-term (up to 14 years post-transplantation, the longest timepoint studied to date) following transplantation into the putamen of PD patients, and despite ongoing disease processes in the host brain.

Cell replacement therapy using DA neurons for PD in the clinic, has so far utilized ventral midbrain DA neurons derived from fetal sources. iPSCs, as described herein, provide several advantages over embryonic stem cells (ESCs) as a cell source for cell replacement in PD and other disorders, including the ability to use patient's own cells or use HLA haplotype-matched cells, and thus eliminate the need for any immune suppression, as patient-specific or HLA-matched cells can be utilized. Immune compatibility is universally known to be very important in all fields of transplantation. Immune suppression is not a trivial matter and may underlie some of the variability previous reported in clinical trials of fetal derived dopamine neuron transplantation (Barker et al., 2013). Indeed, it was previously reported that following the cessation of immunotherapy 6 months after fetal cell transplantation, PD patients lost the benefits of the transplantation (Olanow et al., 2003), and thus, a delayed immune or inflammatory response could have affected the long-term survival, growth, and function of the transplanted dopamine neurons.

### Induced Pluripotent Stem Cells

Induced pluripotent stem (iPS) cell technology has the advantage of providing isogenic cells for cell therapy that limit the patient's immune response. The brain is relatively immunoprivileged but activated microglia can compromise the synaptic function of transplanted neurons (Soderstrom et al., 2008). iPS cells are a type of pluripotent stem cell artificially derived from a non-pluripotent cell, typically an adult somatic cell. This was first demonstrated by Yamanaka et al., who transfected mouse fibroblasts with four genes (Oct4, Sox2, c-Myc, Klf4) to obtain iPS cells in. Subsequently, iPS cells have been derived from human adult somatic cells. (Takahashi et al. Cell, 131:861-872 (2007); Yu et al. Science, 318:1917-1920, 2007).

Depending on the subject to be treated, the iPS cell can be a mammalian cell, for example a mouse, human, rat, bovine, ovine, horse, hamster, dog, guinea pig, or non-human primate cell. For example, if the ultimate goal is to generate therapeutic cells for transplantation into a patient, cells from that patient are desirably used to generate the iPS cells. In one embodiment, the iPS cell is a human iPS cell. In suitable embodiments, the iPS cells used in the present methods are derived from a PD patient. In another embodiment, the iPS cells used in the methods and compositions described herein are derived from a PD patient for autologous treatment.

Somatic cells useful for creating iPS cells can be obtained from any suitable source and can be any differentiated cell type. In certain embodiments, the somatic cells are obtained from blood, synovial fluid or from a tissue. Exemplary somatic cells for reprogramming include, but are not limited to, blood cells, peripheral blood mononuclear cells (PBMC), or fibroblasts.

Somatic cells, as that term is used herein, refer to any cells forming the body of an organism, excluding germline cells. Every cell type in the mammalian body—apart from the sperm and ova, the cells from which they are made (gametocytes) and undifferentiated stem cells—is a differentiated somatic cell. For example, internal organs, skin, bones, blood, and connective tissue are all made up of differentiated somatic cells.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte, a cardiomyocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, immune cells, hepatic, splenic, lung, circulating blood cells, gastrointestinal, renal, bone marrow, and pancreatic cells. In some embodiments, a somatic cell can be a primary cell isolated from any somatic tissue including, but not limited to brain, liver, lung, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

When reprogrammed cells are used for the treatment of Parkinson's disease in a human, it is desirable, but not required, to use somatic cells isolated from the patient being treated.

Essentially any method known in the art can be used for reprogramming somatic cells into iPS cells. When a composition is to be administered to a human, it is preferred that the methods of reprogramming are footprint free and do not make lasting or permanent changes to the genome of the iPS cell, for example, by integrating a nucleic acid overexpressing a particular reprogramming factor. Exemplary methods include reprogramming using modified RNA, plasmids, non-integrating vectors, proteins or small molecules.

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). The resulting cells are referred to as “reprogrammed cells;” when the reprogrammed cells are pluripotent, they are referred to as “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation.

Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors. These methods of re-programming may be preferred for cells to be used for therapeutic purposes, as they are less likely to provoke genomic damage likely to promote, e.g., cancer.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry. Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; b-III-tubulin; a-smooth muscle actin (a-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; b-catenin, and Bmi1. Such cells can also be characterized by the down-regulation of markers characteristic of the somatic cell from which the induced pluripotent stem cell is derived.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

Methods for cell culturing, developing, and differentiating pluripotent stem cells can be carried out with reference to standard literature in the field and are not described in detail herein. Those skilled in the art will appreciate that except where explicitly required otherwise, iPS cells include primary tissue and established lines that bear phenotypic characteristics of iPS cells, and derivatives of such lines that still have the capacity of producing progeny of each of the three germ layers. iPS cell cultures are described as “undifferentiated” or “substantially undifferentiated” when a substantial proportion of stem cells and their derivatives in the population display morphological characteristics of undifferentiated cells, clearly distinguishing them from differentiated cells of embryo or adult origin. Undifferentiated iPS cells are easily recognized by those skilled in the art, and typically appear in the two dimensions of a microscopic view with high nuclear/cytoplasmic ratios and prominent nucleoli. It is understood that colonies of undifferentiated cells within the population will often be surrounded by neighboring cells that are differentiated. Nevertheless, the undifferentiated colonies persist when the population is cultured or passaged under appropriate conditions, and individual undifferentiated cells constitute a substantial proportion of the cell population. Cultures that are substantially undifferentiated contain at least 20% undifferentiated iPS cells, and may contain at least 40%, 60%, or 80% undifferentiated PS cells.

### Differentiation of iPS Cells

In one aspect, the methods for making a population of cells comprising dopaminergic neurons having an A9 phenotype (e.g., expressing FOXA2, b-tubulin and tyrosine hydroxylase) or A10 phenotype provide contacting iPS cells with retinoic acid (RA), an activated form of human SHH, WNT1, and FGF8A in amounts that are sufficient to direct the fate of iPS cells towards dopaminergic neurons, including those preferably having a phenotype of SN-A9 and VTA-A10 DA neurons. In some embodiments, iPS cells are differentiated in the presence of about 10⁻⁹ to 10⁻⁷ M RA (e.g., about 2×10⁹ to about 5×10⁻⁸ M or about 5×10⁻⁹ to about 2×10⁻⁸ M). In a particular embodiment, iPS cells are differentiated in the presence of about 10⁻⁸ M RA. In some embodiments, neuroectodermal differentiation in the presence of RA is allowed to proceed from 10-18 days. In one embodiment, neuroectodermal differentiation in the presence of RA is allowed to proceed for 14 days, with the media changed every 2 days. After differentiation, neuroectodermal colonies are picked and replated for differentiation toward the DA neuron phenotype.

In some embodiments, differentiation toward the DA neuron phenotype occurs by culturing the neuroectodermal colonies with an activated form of human SHH, WNT1, and FGF8A. In some embodiments, the cells are differentiated in the presence of human SHH, WNT1, and FGF8A for 18-35 days. In a particular embodiment, the cells are differentiated in the presence of human SHH, WNT1, and FGF8A for 28 days and then differentiated until Day 49 without SHH, WNT1, and FGF8A, but in the presence of one or more of (e.g., 2, 3, 4, or all 5 of) BDFN, AA, cAMP, GDNF, and TGF-β3.

In some embodiments, the human SHH is an activated form of human SHH. The mature biologically active form of SHH molecule is produced by autocatalytic cleavage of its precursor protein and corresponds to the N-terminal domain of the precursor molecule corresponding generally to residues 24-197 of the full-length human SHH protein (Pepinsky et al, 1998; Taylor et al., 2001). In one embodiment, the effective amount of human SHH protein is an amount sufficient to provide a final concentration in the culture media from about 100 to about 1000 ng/ml. In a particular embodiment, the effective amount of human SHH protein is an amount sufficient to provide a final concentration in the culture media of about 500 ng/ml. In some embodiments, neural progenitor cells are contact with an activated SHH protein. In some embodiments, a supra-potent form of the activated SHH protein can be used. Such modified activated SHH proteins include, for example, activated SHH proteins having N-terminal modifications including, for example, N-terminal acyl amides and thiazolidines, and activated SHH proteins that have been mutagenized to increase biological activity (Taylor et al., 2001). In one embodiment, the modified activated SHH is the C24II SHH which is a 20 kDa protein produced from human cells consisting of 175 amino acid residues, including an N-terminal Ile-Ile sequence substituted for the naturally occurring chemically modified Cys residue (see, Taylor et al., 2001). Other modified activated SHH proteins include, for example C24III, C24IIII, C23IIW, C24IW, C24F, C24I, C24FIF, C24W, C24I-G25I, and C24M (Taylor et al., 2001). When using supra-potent forms of the activated SHH protein, the amount used can be less than the amount of native activated SHH but sufficient to provide the same biological activity as the amounts discussed above. Alternatively, greater amounts can be used in order to deliver higher levels of SHH biological activity to the cells.

In one embodiment, differentiation toward the DA neuron phenotype occurs by culturing the neuroectodermal colonies with FGF8A. FGF8A (fibroblast growth factor 8 A). FGF8 is a member of the fibroblast growth factor family that was originally discovered as a growth factor essential for the androgen-dependent growth of mouse mammary carcinoma cells. Splicing of mouse FGF8 mRNA generates eight secreted isoforms, designated a-h. Only FGF8a, b, e and f exist in humans. FGF8 contains a 22 amino acid (aa) signal sequence, an N-terminal domain that varies according to the isoform (20 aa for FGF8a, which is the shortest), a 125 aa FGF domain and a 37 aa proline rich C-terminal sequence. In some embodiments, the FGF8A is recombinant human FGF8A. In one embodiment, the effective amount of human FGF8A protein is an amount sufficient to provide a final concentration in the culture media from about 10 to about 1000 ng/ml human FGF8A protein. In a particular embodiment, the effective amount of human FGF8A protein is an amount sufficient to provide a final concentration in the culture media of about 100 ng/ml.

In some embodiments, differentiation toward the DA neuron phenotype occurs by culturing the neuroectodermal colonies with human WNT1 protein, either with or without Noggin, and without feeder cells (e.g., MS5 feeder cells). WNT1 has been shown in developmental studies in the embryo to be required to generate appropriately patterned VM neural progenitor cells (Muhr et al., 1999; Nordstrom et al., 2002, 2006). It is also shown herein that neuronal differentiation of FOXA2 neural progenitor cells without exogenous SHH antagonism can occur. While not wishing to be bound by any theory, this result indicates that either SHH antagonists secreted in vitro are sufficient to induce neuronal differentiation or that SHH antagonism is a modest requirement for VM DA neurogenesis in human cells. In some embodiments, the WNT1 is recombinant human WNT1. In one embodiment, the effective amount of human WNT1 protein is an amount sufficient to provide a final concentration in the culture media from about 10 to about 1000 ng/ml human WNT1 protein. In a particular embodiment, the effective amount of human WNT1 protein is an amount sufficient to provide a final concentration in the culture media of about 100 ng/ml.

When differentiated cells are to be administered to humans, it is preferable that the cells are differentiated under conditions that permit the generation of “footprint free,” “xeno-free” or “non-allergenic” cells. For example, care should be taken to avoid differentiation of the human cells in the presence of non-human components, particularly non-human (e.g., mouse) feeder cells. In addition, exposure to differentiation factors, such as WNT1 or SHH, should be transient to ensure that these components can be washed away or diluted out of the culture prior to administration. Thus, in certain embodiments, the differentiation components are not contacted with the neuroectodermal cells using a vector that is capable of inserting a nucleic acid into the genome of the cell. In one embodiment, the methods used to differentiate iPS cells to neuronal cells use a humanized substrate in place of mouse feeder cells. In another embodiment, the methods used to differentiate iPS cells use small molecules or proteins as differentiation factors instead of recombinant or expressed differentiation factors.

### Methods for the Treating of Neurodegenerative Diseases

In the central nervous system, many functionally distinct types of dopaminergic (DA) neurons are found in several brain regions. The cardinal motor symptoms of Parkinson's disease (PD) are caused by the vulnerability of a specific midbrain (SN-A9) type of DA neuron to significant degeneration. The invention provides methods for treating neurodegenerative diseases (e.g., Parkinson's Disease) in a patient by generating dopaminergic neurons, particularly dopaminergic neurons having an A9 phenotype, and transplanting these dopaminergic neurons into the brain of the patient. The dopaminergic neurons are generated, for example, from neuronal progenitor cells by contacting those cells with an effective amount of retinoic acid, human sonic hedgehog (SHH) protein, WNT1 protein, and FGF8A protein. Optionally, the neuronal progenitor cells can be generated by contacting PS cells with RA and other differentiating factors under culture conditions described herein.

Transplantation can be allogeneic (between genetically different members of the same species), autologous (transplantation of an organism's own cells or tissues), syngeneic (between genetically identical members of the same species (e.g., identical twins)), or xenogeneic (between members of different species). Ordinarily, the DA neurons would be transplanted into the substantia nigra (particularly in or adjacent of the compact region), the ventral tegmental area (VTA), the caudate, the putamen, the nucleus accumbens, the subthalamic nucleus, or any combination thereof, of the brain to replace the DA neurons whose degeneration resulted in PD. Transplantation into the substantia nigra, the caudate, or the putamen is performed because, although the cell bodies of A9 DA neurons are located in the substantia nigra, their axons extend into the forebrain structures where dopamine release occurs. In disease conditions where it is desirable to replace A10 DA neurons, the DA neurons are transplanted into the VTA, the nucleus accumbens, or both regions of the brain. In the late stages of PD, cognitive and behavioral disturbance can be generated from DA loss and synaptic dysfunction in the caudate, cerebral cortex deep layers, nucleus accumbens, and substantia nigra regions of the brain. Thus, the ventral tegmental DA neuronal phenotype of A10 would be specifically transplanted to these regions to replace lost A10 DA functions. In particular, transplantation of A10 DA neurons, or cells primed to differentiate into A10 DA neurons, to the caudate nucleus would be the most effective replacement.

Transplantation of the transplant composition as described herein into the brain of the patient with a neurodegenerative disease results in replacement of lost, non-, or dysfunctional DA neurons. The cells are introduced into a subject with Parkinson's disease in an amount suitable to replace the dysfunctional DA neurons such that there is an at least partial reduction or alleviation of at least one adverse effect/symptom of the disease or permit a reduction in dose of pharmacological treatments of the disease. The cells can be administered to a subject by any appropriate route that results in delivery of the cells to a desired location in the subject where at least a portion of the cells remain viable. Typically, the transplant composition is injected in the caudate, putamen, nucleus accumbens, or the subthalamic nucleus by stereotactic injection. It is preferred that at least about 5%, preferably at least about 10%, more preferably at least about 20%, yet more preferably at least about 30%, still more preferably at least about 40%, and most preferably at least about 50% or more of the cells remain viable after administration into a subject. The period of viability of the cells after administration to a subject can be as short as a few hours, e.g., twenty-four hours, to a few days, to as long as a few weeks to months. One transplantation method that can be used to deliver the cells to a subject is described by Bjorklund et al. (Proc. Nat. Acad. Sci. USA, 99:2344-2349, 2002).

To accomplish these methods of administration, the cells of the invention can be inserted into a delivery device that facilitates introduction by injection (e.g., stereotactic injection) or implantation of the cells into the subject. Typically, the cells are injected into the target area as a cell suspension. Alternatively, the DA neurons can be embedded in a solid or semisolid support matrix when contained in such a delivery device.

Cell transplantation therapies typically involve the intraparenchymal (e.g., intracerebral) grafting of the replacement cell populations into the lesioned region of the nervous system, or at a site adjacent to the site of injury. Most commonly, the therapeutic cells are delivered to a specific site by stereotaxic injection. Conventional techniques for grafting are described, for example, in Bjorklund et al. (Neural Grafting in the Mammalian CNS, eds. Elsevier, pp. 169-178, 1985), Leksell et al. (Acta Neurochir., 52:1-7, 1980) and Leksell et al. (J. Neurosurg., 66:626-629, 1987). Identification and localization of the injection target regions will generally be done using a non-invasive brain imaging technique (e.g., MRI) prior to implantation (see, for example, Leksell et al., J. Neurol. Neurosurg. Psychiatry, 48:14-18, 1985). Briefly, administration of cells into selected regions of a patient's brain can be made by drilling a hole and piercing the dura to permit the needle of a microsyringe to be inserted. Alternatively, the cells can be injected into the brain ventricles or intrathecally into a spinal cord region. It also is possible to effect multiple grafting concurrently, at several sites, using the same cell suspension, as well as mixtures of cells. Multiple graftings or sites of injection are particularly useful for administration of cells to larger brain structures such as the caudate and/or putamen.

### Therapeutic Compositions

Following in vitro cell culture and isolation as described herein, the cells are prepared for implantation. The cells are suspended in a physiologically compatible carrier, such as cell culture medium (e.g., Eagle's minimal essential media), phosphate buffered saline, or artificial cerebrospinal fluid (aCSF). The volume of cell suspension to be implanted will vary depending on the site of implantation, treatment goal, and cell density in the solution. For the treatment of Parkinson's Disease, about 30-100 μl of cell suspension will be administered in each intra-nigral or intra-putamenal injection and each patient may receive a single or multiple injections into each of the left and right nigral or putaminal regions.

Pharmaceutically acceptable carriers and diluents include saline, aqueous buffer solutions, solvents and/or dispersion media. The use of such carriers and diluents is well known in the art. The solution is preferably sterile and fluid. Suitably, the solution is stable under the conditions of manufacture and storage and preserved against the contaminating action of microorganisms such as bacteria and fungi through the use of, for example, parabens, chlorobutanol, phenol, ascorbic acid, or thimerosal. Solutions of the invention can be prepared by incorporating the cells as described herein in a pharmaceutically acceptable carrier or diluent and, as required, other ingredients.

In some embodiments, the cells are encapsulated within permeable membranes prior to implantation. Encapsulation provides a barrier to the host's immune system and inhibits graft rejection and inflammation. Several methods of cell encapsulation may be employed. In some instances, cells will be individually encapsulated. In other instances, many cells will be encapsulated within the same membrane. Several methods of cell encapsulation are well known in the art, such as described in European Patent Publication No. 301,777, or U.S. Pat. Nos. 4,353,888, 4,744,933, 4,749,620, 4,814,274, 5,084,350, and 5,089,272.

In one method of cell encapsulation, the isolated cells are mixed with sodium alginate and extruded into calcium chloride so as to form gel beads or droplets. The gel beads are incubated with a high molecular weight (e.g., MW 60-500 kDa) concentration (0.03-0.1% w/v) polyamino acid (e.g., poly-L-lysine) to form a membrane. The interior of the formed capsule is re-liquefied using sodium citrate. This creates a single membrane around the cells that is highly permeable to relatively large molecules (MW ˜200-400 kDa), but retains the cells inside. The capsules are incubated in physiologically compatible carrier for several hours in order that the entrapped sodium alginate diffuses out and the capsules expand to an equilibrium state. The resulting alginate-depleted capsules is reacted with a low molecular weight polyamino acid which reduces the membrane permeability (MW cut-off ^{˜}40-80 kDa).

Prior to introduction into a subject, the DA neurons can be modified to inhibit immunological rejection. For example, to inhibit rejection of transplanted cells and to achieve immunological non-responsiveness in a transplant recipient, the methods of the invention can include alteration of immunogenic antigens on the surface of the cells prior to introduction into the subject. This step of altering one or more immunogenic antigens on the cells can be performed alone or in combination with administering to the subject an agent that inhibits T cell activity in the subject. Alternatively, inhibition of rejection of the transplanted cells can be accomplished by administering to the subject an agent that inhibits T cell activity in the subject in the absence of prior alteration of an immunogenic antigen on the surface of the transplanted cells. An agent that inhibits T cell activity is defined as an agent, which results in removal (e.g., sequestration) or destruction of T cells within a subject or inhibits T cell functions within the subject. T cells may still be present in the subject but are in a non-functional state, such that they are unable to proliferate or elicit or perform effector functions (e.g., cytokine production, cytotoxicity, etc.). The agent that inhibits T cell activity may also inhibit the activity or maturation of immature T cells (e.g., thymocytes). A suitable agent for use in inhibiting T cell activity in a recipient subject is an immunosuppressive drug that inhibits or interferes with normal immune function, e.g., cyclosporin A, FK506, or RS-61443. Additional therapeutic agents that can be administered include steroids (e.g., glucocorticoids such as prednisolone, methyl prednisolone, and dexamethasone).

The inventors have shown that a threshold number of cells are required to achieve appropriate engraftment of dopaminergic neurons and regression or elimination of symptoms. For example, the inventors have found that 13,000 dopaminergic neurons can reduce or eliminate Parkinson's symptoms in a primate animal model. Accordingly, in one embodiment, the transplantation composition comprises at least 7000 dopaminergic neurons expressing FOXA2, tyrosine hydroxylase and β-tubulin (e.g., having an A9 phenotype). In other embodiments, the transplantation composition comprises at least 8000, at least 9000, at least 10,000, at least 11,000, at least 12,000, at least 13,000, at least 14,000, at least 15,000, at least 16,000, at least 17,000, at least 18,000, at least 19,000, at least 20,000, at least 25,000, at least 30,000, at least 35,000, at least 40,000, at least 45,000, at least 50,000, at least 55,000, at least 60,000, at least 65,000, at least 70,000, at least 75,000, at least 80,000, at least 85,000, at least 90,000, at least 95,000, at least 100,000 dopaminergic neurons expressing FOXA2, tyrosine hydroxylase and β-tubulin.

It will be appreciated by one of skill in the art that a transplantation composition useful for treating and/or preventing Parkinson's disease does not need to be a pure, homogeneous culture of dopaminergic neurons expressing FOXA2, tyrosine hydroxylase and β-tubulin (data not shown). Accordingly, in one embodiment, the transplantation composition comprises at least 2% dopaminergic neurons expressing FOXA2, tyrosine hydroxylase and β-tubulin. In other embodiments, the transplantation composition comprises at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more dopaminergic neurons expressing FOXA2, tyrosine hydroxylase and β-tubulin.

The inventors have found that transplantation compositions comprising an impure mixture of dopaminergic neurons expressing FOXA2, tyrosine hydroxylase and β-tubulin with other cellular components have better engraftment of the dopaminergic neurons upon transplantation, and result in better treatment and reduction of symptoms of Parkinson's disease (data not shown). Accordingly, in some embodiments the transplantation composition comprises less than 75%, less than 70%, less than 65%, less than 60%, less than 55%, less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, or less than 2% dopaminergic neurons expressing FOXA2, tyrosine hydroxylase and β-tubulin among other cellular components. In certain embodiments, the transplantation composition comprises dopaminergic neurons expressing FOXA2, tyrosine hydroxylase and β-tubulin in the range of 1-60%, 1-50%, 1-40%, 1-30%, 1-20%, 1-10%, 1-5%, 20-60%, 30-60%, 40-60%, 10-60%, 10-50%, 10-40%, 10-30%, 10-20%, 20-60%, 20-50%, 20-40%, 20-30%, 30-60%, 30-50%, 30-40%, 40-60%, 40-50%, 5-30%, 5-20%, 5-10%, 2-4%, 4-6%, 10-20%, 30-40%, 40-50%, 50-60%, or any range therebetween.

The inventors have also found that there is a threshold amount of dopaminergic neuron engraftment or synapse formation that is required in order to reduce or eliminate the symptoms of Parkinson's disease (data not shown). Typically, Parkinson's symptoms do not occur until the disease reaches an advanced stage and approximately 60% of the dopaminergic neurons are dysfunctional or dead, which means that less than 40% of the cells are functioning. Thus, the transplantation composition aims to reduce or eliminate symptoms of Parkinson's disease by, e.g., increasing the total number of functional dopaminergic neurons to at least 45%. In other embodiments, the transplantation compositions is calibrated to administer a number of cells useful for increasing the number of functional dopaminergic neurons to at least 50%, at least 60%, at least 70%, at least 75%, at least 80%, at least 85%, at least 90%, at least 95%, at least 98%, at least 99%, or even 100% of the dopaminergic neurons. The inventors have also found that the symptoms of Parkinson's disease are not reduced until a functional number of synapses are formed. In some embodiments, it is useful to calibrate the number of cells to be administered based on the number on the number of cells required to permit formation or reformation of at least 40% of the synapses that would exist in the brain region in a normal subject. For example, symptoms of Parkinson's disease are not reduced or alleviated until there are functional synapses or neurons at approximately 40-50% of the cells or synapses normally observed in the brain. Thus, in certain embodiments, the transplantation composition is calibrated to achieve the formation of at least 60%, at least 70%, least 80%, at least 90%, at least 95%, at least 98%, at least 99%, or even 100% of the functional synapses observed in a normal brain.

In certain embodiments, the transplant composition further comprises a factor(s) that can improve engraftment or improve survival or health of cells in the composition. Such factors can be a growth factor, a neurotrophic factor, a neuropoietic cytokine, or one or more differentiation factors. When cells are to be administered to humans, such factors should be xeno-free. In one embodiment, a neurotrophic factor is added to the transplant composition. Exemplary neurotrophic factors include, but are not limited to, gliglial cell line-derived neurotrophic factor (GDNF), neurturin (NRTN), artemin (ARTN), and persephin (PSPN). Exemplary neurotrophins for use in the methods and compositions described herein include, but are not limited to, nerve growth factor (NGF), brain-derived neurotrophic factor (BDNF), neurotrophin-3 (NT-3), and neurotrophin-4 (NT-4).

When the transplant composition is to be administered to a human, the culture comprising dopaminergic neurons differentiated from iPS cells should be depleted of cells that are capable of forming a tumor or teratoma. Depletion of such cells can be performed by removing cells from the culture that express one or more stem cell markers. In one embodiment, the culture is depleted of cells that express SSEA and/or TRA160.

Given the intricate nature of administering a transplant composition to a specific region of the brain, the inventors have found that the transplant composition should be a small volume, in part, to minimize localized damage to the brain. The composition can be administered in more than one site (e.g., 1, 2, 3, 4, 5, 6, 7, 8, 9, or 10) and a volume can be injected two or more times in each site. Accordingly, in some embodiments, the transplant composition is in a volume of less than 2 mL, less than 1.75 mL, less than 1.5 mL, less than 1.25 mL, less than 1.0 mL, less than 0.9 mL, less than 0.8 mL, less than 0.7 mL, less than 0.6 mL, less than 0.5 mL, less than 0.4 mL, less than 0.3 mL, less than 0.2 mL, less than 0.1 mL or smaller.

### Cell Transplantation

Transplantation can be allogeneic (between genetically different members of the same species), autologous (transplantation of an organism's own cells or tissues), syngeneic (between genetically identical members of the same species (e.g., identical twins)), or xenogeneic (between members of different species). In some embodiments, autologous transplantation is preferred to avoid the need for immunosuppressive agents. Typically, dopaminergic neurons are transplanted into the substantia nigra, the ventral tegmental area (VTA), the caudate, the putamen, the nucleus accumbens, the subthalamic nucleus, or any combination thereof, of the brain to replace the dopaminergic neurons whose degeneration resulted in Parkinson's disease (PD). Transplantation into the substantia nigra, the caudate, or the putamen is performed because, although the cell bodies of A9 dopaminergic (DA) neurons are located in the substantia nigra, their axons extend into the forebrain structures where dopamine release occurs. In most cases it is desirable to transplant a composition comprising A9 DA neurons, as the engraftment of A9 DA cells have been shown to have the best outcome in rodent models of Parkinson's disease with regard to symptom reduction etc.

In disease conditions where it is desirable to replace A10 DA neurons, the DA neurons (obtained from in vitro differentiation of ES cells) are transplanted into the VTA, the nucleus accumbens, or both regions of the brain. In the late stages of PD, cognitive and behavioral disturbance may be generated from DA loss and synaptic dysfunction in the caudate, cerebral cortex deep layers, nucleus accumbens, and substantia nigra regions of the brain. Thus, the ventral tegmental DA neuronal phenotype of A10 would be specifically transplanted to these regions to replace lost A10 DA functions. In particular, transplantation of A10 DA neurons, or cells primed to differentiate into A10 DA neurons, to the caudate nucleus would be the preferred and most effective replacement.

Transplantation of a compositions as described herein into the brain of the patient with a neurodegenerative disease results in replacement of lost, non-, or dysfunctional DA neurons. The composition is introduced into a subject having Parkinson's disease in an amount suitable to replace the dysfunctional DA neurons such that there is an at least partial reduction or alleviation of at least one adverse effect or symptom of the disease. The cells can be administered to a subject by any appropriate route that results in delivery of the cells to a desired location in the subject where at least a portion of the cells remain viable. It is preferred that at least 5% remain viable. In other embodiments, at least 10%, at least 20%, at least 30%, at least 40%, or at least 50%, at least 60%, at least 70%, at least 80%, at least 90%, at least 95%, at least 98%, at least 99% or more of the cells remain viable after administration into a subject. The period of viability of the cells after administration to a subject can be as short as a few hours, e.g., twenty-four hours, to a few days, to as long as a few weeks to months. One transplantation method that can be used to deliver the composition comprising dopaminergic neurons to a subject is described by Bjorklund et al. (Proc. Nat. Acad. Sci. USA 99:2344-2349, 2002). The DA neurons can be administered in a physiologically compatible carrier, such as a buffered saline solution. To treat disorders characterized by neuron degeneration in a human subject, a population of DA neurons as a suspension of 25,000 to 250,000 cells per microliter in a pharmaceutically acceptable carrier, such that the cells form, in the patient, a population of cells in which at least 5%, 10%, 20%, 30%, 40%, 50%, 60%, 70%, 80%, or 90% of the cells are dopaminergic, is introduced into the patient. Preferably, the suspension is 50,000 to 200,000 cells per microliter. More preferably, the suspension is 75,000 to 150,000 cells per microliter.

In some embodiments, the subject is first diagnosed as having Parkinson's disease prior to administering the transplant composition according to the methods described herein (e.g., assessment using UPDRS or PET scanning) In some embodiments, the subject is first diagnosed as being at risk of developing Parkinson's disease prior to administering the cells (e.g., a subject having a family history of Parkinson's disease or a genetic pattern indicating a predisposition to Parkinson's disease).

In some embodiments of the aspects described herein, the cells of the transplant compositions are expanded in culture prior to differentiation to dopaminergic neurons and administration to a subject in need thereof.

To accomplish these methods of administration, the transplant composition can be inserted into a delivery device that facilitates introduction by injection or implantation of the cells into the subject. Typically, the cells are injected into the target area as a cell suspension. Alternatively, the DA neurons can be embedded in a solid or semisolid support matrix when contained in such a delivery device.

The solution includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, or thimerosal. Solutions for use with the methods and compositions described herein can be prepared by incorporating the cells as described herein in a pharmaceutically acceptable carrier or diluent and, as required, other ingredients.

Support matrices in which the DA neurons as described herein can be incorporated or embedded include matrices which are recipient-compatible and which degrade into products that are not harmful to the recipient. Natural and/or synthetic biodegradable matrices are examples of such matrices. Natural biodegradable matrices include, for example, collagen matrices. Synthetic biodegradable matrices include synthetic polymers such as polyanhydrides, polyorthoesters, and polylactic acid. These matrices provide support and protection for the cells in vivo.

In some embodiments, the transplantation method is repeated after a given interval of time to permit the original transplantation to engraft (e.g., at least 3 months after the original treatment, at least 4 months, at least 5 months, at least 6 months, at least 1 year, at least 18 months, at least 2 years, at least 3 years or more). Repeated treatments can be performed, for example, to permit the threshold level of engraftment necessary to reduce or eliminate Parkinson's disease. In some embodiments, the method is repeated twice, three times, four times, five times or more.

### Efficacy Measurement

The term “effective amount” as used herein refers to the amount of a population of dopaminergic neurons expressing FOXA2, β-tubulin and tyrosine hydroxylase (TH) needed to alleviate at least one or more symptoms of Parkinson's disease, and relates to a sufficient amount of a composition to provide the desired effect. An effective amount as used herein would also include an amount sufficient to prevent or delay the development of a symptom of the disease, alter the course of a symptom of the disease (for example but not limited to, slow the progression of a symptom of the disease, such as difficulty in initiating movement), or reverse a symptom of the disease. It is understood that for any given case, an appropriate “effective amount” can be determined by one of ordinary skill in the art using routine experimentation. Given the intricacies of the brain and the unpredictable nature of cell engraftment, the “effective amount” of cells may vary among different patients, however one can easily determine in hindsight if the amount of cells administered was indeed an ‘effective amount.” Thus, further treatments can be modified accordingly.

The efficacy of treatment can be determined by the skilled clinician. However, a treatment is considered “effective treatment,” as the term is used herein, if any one or all of the symptoms, or other clinically accepted symptoms or markers of Parkinson's disease are reduced, e.g., by at least 10% following treatment with a composition comprising dopaminergic neurons expressing FOXA2, β-tubulin, and tyrosine hydroxylase as described herein. Methods of measuring these indicators are known to those of skill in the art and/or described herein.

Indicators of Parkinson's disease include, increased time to initiate movement, tremor, stiff muscles, difficulty standing, difficulty walking, involuntary movements, muscle rigidity, impaired coordination, shuffling gait, dizziness, poor balance, amnesia, confusion, reduced facial expressions, difficulty swallowing, drooling, unintentional writhing, among others.

In one embodiment, effective treatment is determined by a reduction in the dose of pharmacological treatments, such as L-DOPA, required to maintain adequate control of symptoms of Parkinson's disease.

In one embodiment, efficacy of treatment is monitored using the Unified Parkinson's Disease Rating Scale (UPDRS).

The present invention may be as defined in any one of the following numbered paragraphs:

1. A composition for the treatment and/or prevention of Parkinson's Disease, the composition comprising: (a) a population of cells comprising at least 3% dopaminergic neurons, wherein the dopaminergic neurons express FOXA2, β-tubulin, and tyrosine hydroxylase (TH), (b) a neurotrophic factor, and (c) a pharmaceutically acceptable carrier.

2. The composition of paragraph 1, wherein the neurotrophic factor is selected from the group consisting of: glial cell line-derived neurotrophic factor (GDNF), neurturin (NRTN), artemin (ARTN), and persephin (PSPN).

3. The composition of paragraph 1 or 2, wherein the neurotrophic factor is GDNF.

4. The composition of paragraph 1, 2, or 3, wherein the population of cells comprises at least 20% dopaminergic neurons.

5. The composition of any one of paragraphs 1-4, wherein the population of cells comprises between 4-40% dopaminergic neurons.

6. The composition of any one of paragraphs 1-5, wherein the population of cells is free of teratoma-forming cells.

7. The composition of any one of paragraphs 1-6, wherein the teratoma-forming cells express TRA60 and/or SSEA.

8. The composition of any one of paragraphs 1-7, wherein the pharmaceutically acceptable carrier is artificial cerebrospinal fluid.

9. The composition of any one of paragraphs 1-8, wherein the dopaminergic neurons are post-mitotic cells.

10. The composition of any one of paragraphs 1-9, wherein the volume of the composition is less than 1 mL.

11. The composition of any one of paragraphs 1-10, wherein the population of cells comprises at least 10,000 dopaminergic neurons.

12. The composition of any one of paragraphs 1-11, wherein the composition is xeno-free.

13. The composition of any one of paragraphs 1-12, wherein the dopaminergic neurons are footprint free.

14. The composition of any one of paragraphs 1-13, wherein the dopaminergic neurons further express G-protein inwardly-rectifying potassium channel (GIRK)-2.

15. A method for treating and/or preventing Parkinson's disease, the method comprising: administering a composition of paragraph 1 into the caudate, putamen, nucleus accumbens, or subthalamic nucleus of a subject in need thereof, thereby treating and/or preventing Parkinson's disease.

16. The method of paragraph 15, wherein the composition is administered to the putamen.

17. The method of paragraph 15 or 16, wherein the composition is administered bilaterally.

18. The method of paragraph 15, 16, or 17, wherein the composition is administered using stereotactic injection.

19. The method of any one of paragraphs 15-18, wherein the subject is diagnosed as having Parkinson's disease and/or is monitored following transplantation by positron emission tomography (PET) scanning.

20. The method of any one of paragraphs 15-19, wherein the subject is diagnosed or monitored for successful transplantation using the Unified Parkinson's Disease Rating Scale (UPRDS).

21. The method of any one of paragraphs 15-20, wherein the subject is a human.

22. The method of any one of paragraphs 15-21, wherein the method is repeated.

23. The method of paragraph 22, wherein the method is repeated until the subject is substantially free of Parkinson's disease symptoms.

24. The method of paragraph 22, wherein the method restores the at least 60% of the functional dopaminergic synapses normally observed in the putamen.

25. The method of any one of paragraphs 15-24, wherein the method reduces the dose of L-DOPA required by the subject to achieve adequate clinical control of symptoms.

26. A method for reducing the dose of L-DOPA required by a Parkinson's patient to achieve adequate clinical control of symptoms, the method comprising: (a) administering a composition of paragraph 1 into the caudate, putamen, nucleus accumbens, or subthalamic nucleus of a subject receiving L-DOPA to control Parkinson's symptoms, (b) monitoring the subject for engraftment of dopaminergic neurons and/or for side effects associated with L-DOPA, and (c) reducing or eliminating the dose of L-DOPA as required to reduce L-DOPA associated side effects while maintaining adequate clinical control of Parkinson's symptoms.

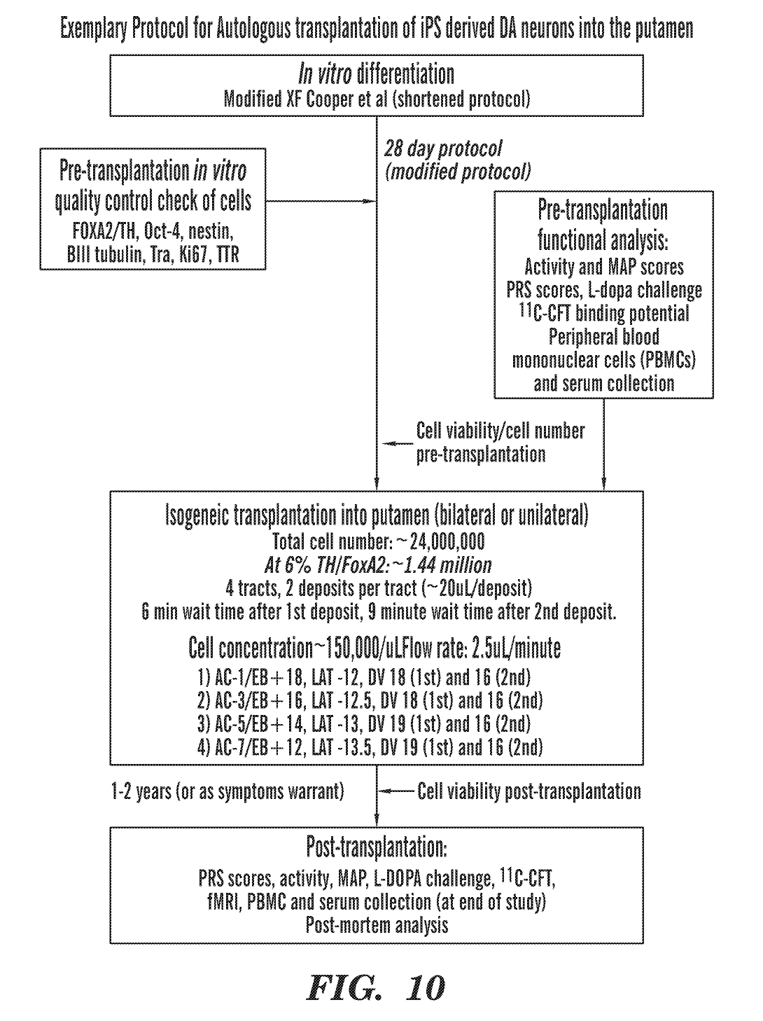

27. A method of treating and/or preventing Parkinson's disease, the method comprising the steps outlined in FIG. 10.

28. A method comprising: (a) reprogramming somatic cells obtained from a subject into iPS cells, (b) contacting the iPS cells with retinoic acid, human SHH and FGF8A to promote differentiation of the iPS cells into dopaminergic neurons, wherein the dopaminergic neurons express FOXA2, β-tubulin and tyrosine hydroxylase, and (c) depleting the population of cells of step (b) of teratoma-forming cells.

29. The method of paragraph 28, wherein the population of cells of step (c) comprises at least 4% dopaminergic neurons that express FOXA2, β-tubulin and tyrosine hydroxylase.

30. The method of paragraph 28 or 29, wherein the dopaminergic neurons further express GIRK2.

31. The method of paragraph 28, 29, or 30, wherein the teratoma-forming cells are depleted from the population of cells of step (b) by removing cells expressing TRA160 and/or SSEA.

32. The method of any one of paragraphs 28-31, wherein the somatic cells are peripheral blood mononuclear cells (PBMCs) or fibroblasts.

33. The method of any one of paragraphs 28-32, further comprising a step of freezing and thawing the population of cells of step (b) or step (c).

34. The method of any one of paragraphs 28-33, further comprising admixing the depleted culture of step (c) with a pharmaceutically acceptable carrier.

35. Use of a composition of any one of paragraphs 1-14 for the treatment and/or prevention of Parkinson's disease.

### EXAMPLES

### Example 1: Successful Function of Autologous iPSC-Derived Dopamine Neurons Following Transplantation in a Non-Human Primate Model of Parkinson's Disease

### SUMMARY

Autologous transplantation of patient-specific induced pluripotent stem cell (iPSC)-derived neurons is a potential clinical approach for treatment of neurological disease. Preclinical demonstration of long-term efficacy, feasibility, and safety of iPSC-derived dopamine neurons in non-human primate models is an important step in clinical development of cell therapy. Here, cynomolgus monkeys (CM) were used as a Parkinson's disease (PD) model and were analyzed for up to 2 years following autologous transplantation with iPSC-derived midbrain dopamine neurons. In one animal, with the most successful protocol, it was found that unilateral engraftment of CM-iPSCs provided a gradual onset of functional motor improvement contralateral to the side of dopamine neuron transplantation, and increased motor activity, without a need for immunosuppression. Postmortem analyses demonstrated robust survival of midbrain-like dopaminergic neurons and extensive outgrowth into the transplanted putamen.

### RESULTS

Cellular therapies offer an exciting opportunity to replace specific populations of cells in neurodegenerative diseases where symptoms are defined by the loss of a specific cell type, such as the degeneration of substantia nigra (SN) dopamine neurons in Parkinson's disease (PD). The use of induced pluripotent stem cell (iPSC)-derived neurons as an autologous cell source overcomes the current limitations posed by allogeneic donor cells in PD. Fetal ventral midbrain allografts can survive and function in the human PD brain for over 18 years (C. R. Freed et al., 2013, Soc. Neurosci., abstract; Hallett et al., 2014; Kefalopoulou et al., 2014; Mendez et al., 2005; Politis et al., 2010); however, such techniques will never become an easily accessible therapeutic option for patients due to the requirement of fetal donor tissue from elective abortions. Allografting in the brain also creates a greater immune reaction over time compared with isogeneic grafting (Duan et al., 1995; Morizane et al., 2013). The generation of midbrain-like dopamine neurons from patient-specific iPSCs and subsequent autologous transplantation is a rational long-term strategy for cell replacement in PD. Previous reports of autologous transplantation in a non-human primate PD model have demonstrated the advantage of autologous versus allogeneic grafts and shown dopamine neuron survival in the primate brain for up to 6 months to 1 year after transplantation (Emborg et al., 2013; Morizane et al., 2013; Sundberg et al., 2013). However, the long-term function, survival, and safety of iPSC-derived dopamine neurons following autologous transplantation in a non-human primate model of PD has not yet been established.

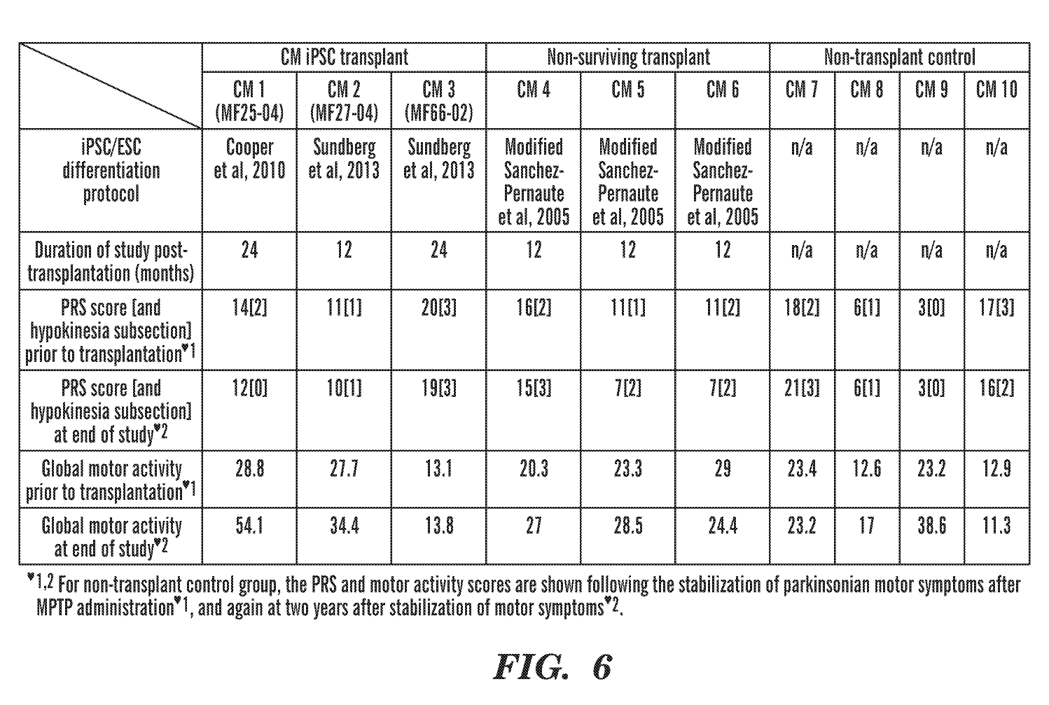

All studies were approved by the Harvard Medical School Institutional Animal Care and Use Committee (IACUC). To induce parkinsonism in cynomolgus monkeys (CMs), systemic low-dose 1-methyl-4-phenyl-1,2,3,6-tetrahydropyridine (MPTP) was administered, which resulted in a progressive and persistent reduction in global motor activity and a stable bilateral parkinsonian syndrome, including tremor, rigidity, bradykinesia, hypokinesia, posture/balance disturbances, and impairment in both gross and fine motor skills (FIG. 6) (Brownell et al., 1998a; Hantraye et al., 1992; Wullner et al., 1994). All animals displayed a significant loss of dopamine transporters (DATs) in the putamen as measured by ¹¹C-(2b-carbomethoxy-3b-(4-fluorophenyl) tropane) (¹¹C-CFT) binding potential, as previously described (Brownell et al., 1998a). In a successive series of studies, three MPTP-lesioned CMs (MF25-04, MF66-02, and MF27-04) received autologous transplantation of CM-iPSCderived neural cells into the putamen in order to assess the function and survival of engrafted autologous iPSC-derived dopamine neurons. CM-iPSCs from MF25-04 were differentiated using the protocol of Cooper et al. (2010), and CM-iPSCs from MF27-04 and MF66-02 were differentiated using the protocol of Sundberg et al. (2013). No animals in this study received any immunosuppression for the duration of the study.

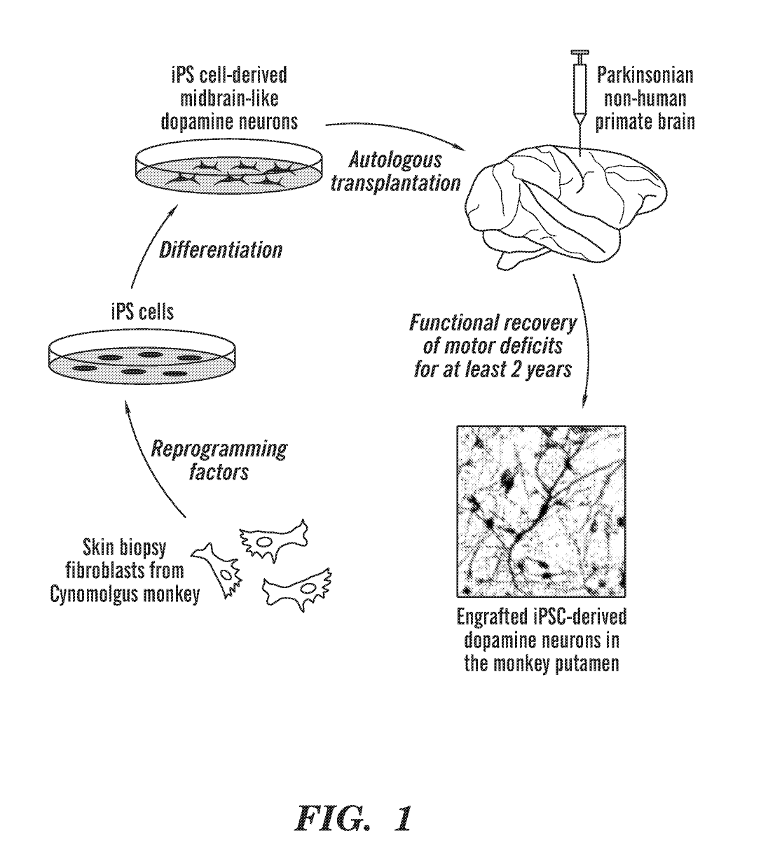

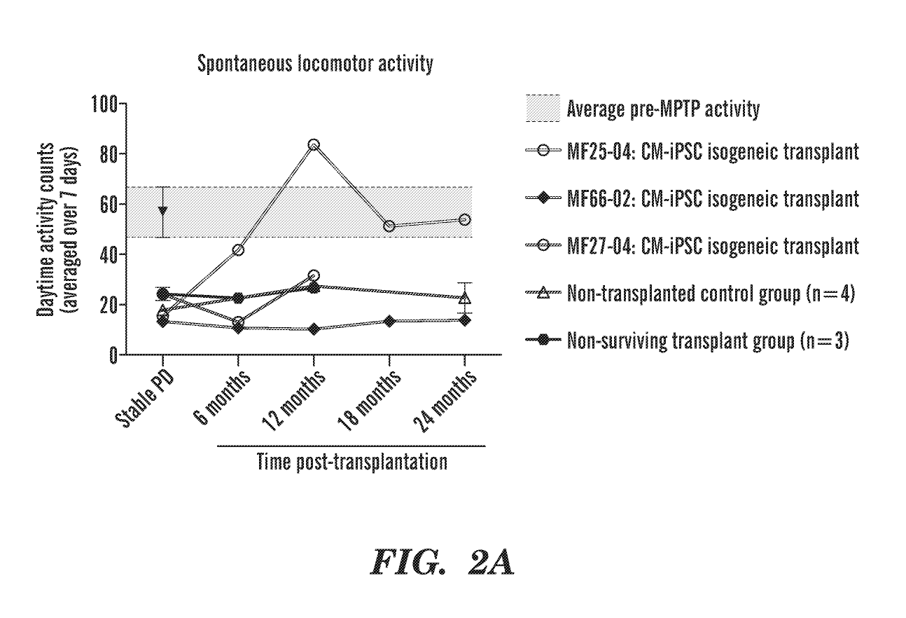

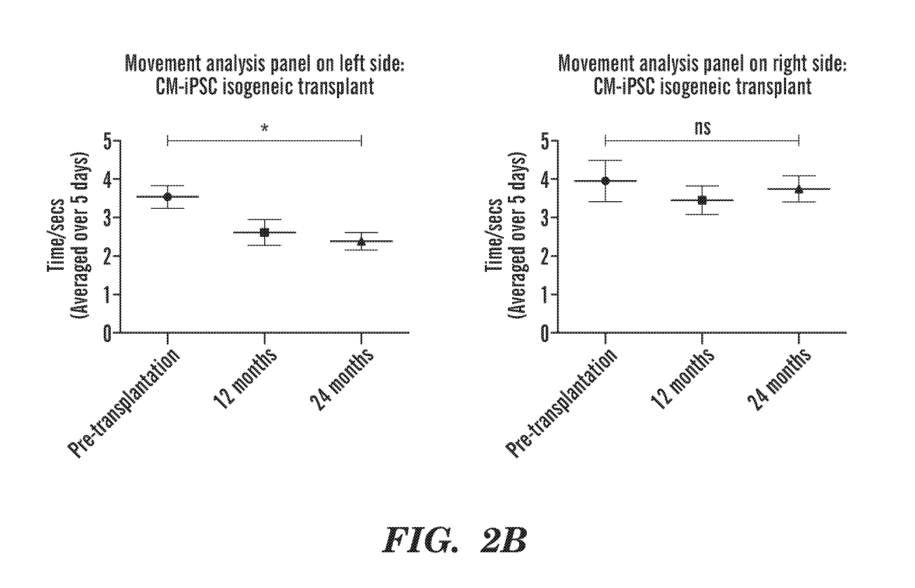

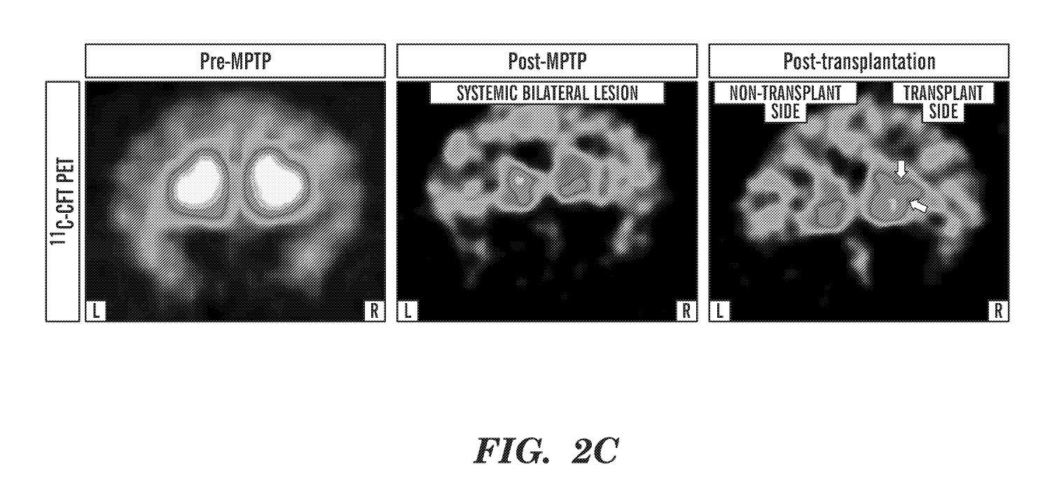

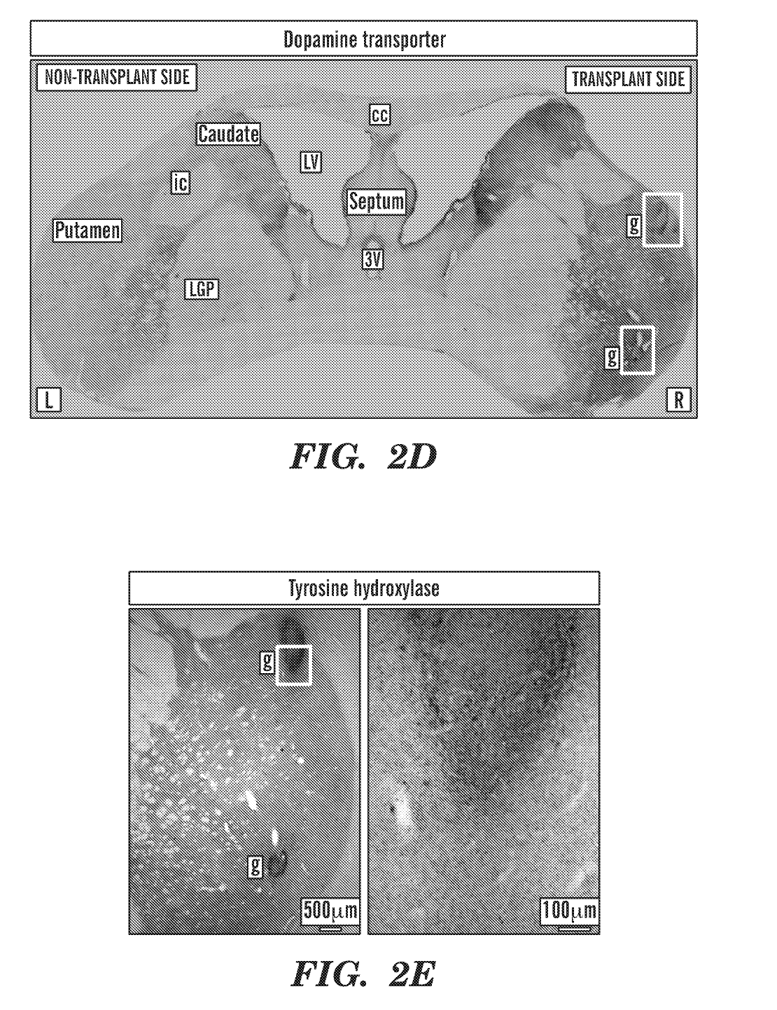

Global daytime motor activity was measured in MPTP-lesioned CMs that had received autologous transplantation of iPSC-derived neural cells, using an automated activity monitor (FIG. 1A). At 6 months after transplantation, daytime activity counts in animal MF25-04 (autologous iPSC transplant) were (contralateral to the graft) upper limb motor function, compared with baseline values, was observed by 12 months after transplantation in MF25-04, and this reduction reached significance at 24 months after transplantation (p<0.05, one-way ANOVA). Use of right upper limb motor function in MF25-04 was not significantly altered over the 24 months following the transplantation procedure. The severity of parkinsonian signs was also rated monthly using a parkinsonian rating scale (FIG. 6). A reduction in the hypokinesia subsection of the parkinsonian rating scale in MF25-04 was observed; from a score of 2 prior to transplantation (maximum possible score is 3) to a score of 0 at 12 months after transplantation, and this remained stable at 0 until completion of the study (Table 1). Overall, the time course of functional recovery observed in MF25-04 was consistent with the developmental maturation, outgrowth, and connectivity of analogous fetal non-human primate dopamine neurons (Redmond et al., 2008; Tsui and Isacson, 2011). No marked changes in global motor activity, MAP test time, or hypokinesia were observed in animals MF27-04 and MF66-02 at 1-2 years after autologous transplantation of differentiated CM-iPSCs (FIG. 2A; FIG. 6), indicating insufficient survival of engrafted CM-iPSC-derived midbrain dopamine neurons and reinnervation of the transplanted putamen (Grealish et al., 2010; Redmond et al., 2008). Motor behavior was also analyzed in MPTP-lesioned CMs that received allogeneic transplantation with differentiated primate embryonic stem (ES) cells (Cyno-1) with no immunosuppression, as previously described (Sanchez-Permute et al., 2005), in which less than 50 surviving dopaminergic neurons were detected in the grafted putamen at postmortem (termed the “non-surviving transplant” group) (n=3), and also in non-transplanted MPTP-lesioned CMs (n=4) (FIG. 6). In the non-surviving transplant group, animals were followed for 12 months after the transplantation procedure before termination of the study; no significant changes in motor behavior were observed in these animals during this time (FIG. 2A). Motor behavior was also not altered over a 2-year period in the non-transplant control group of parkinsonian animals (FIG. 2A), consistent with the long-term functional stability of this bilateral MPTP non-human primate model (Brownell et al., 1998a; Hantraye et al., 1992; Wullner et al., 1994).

Since functional improvement in parkinsonian motor symptoms was observed in the MF25-04 iPSC autologous transplant case, performed ¹¹C-CFT-PET scanning was performed to assess dopamine nerve terminals in the caudate and putamen at 2 years after transplantation of CM-iPSC-derived neural cells (Brownell et al., 1998a; Hantraye et al., 1992; Jenkins et al., 2004). At 2 years after transplantation, a marked increase in ¹¹C-CFT binding sites was observed in the right putamen compared with the non-transplanted left putamen, indicative of functional dopaminergic neurons on the transplanted side (FIG. 2C).

Given the motor improvement and positive neuroimaging indicative of a functional graft in MF25-04, extensive postmortem examination of graft survival and morphology was performed at 2 years after transplantation. Macroscopic examination of the brain showed graft deposit sites within the putamen and normal striatal cytoarchitecture, with no displacement or compression of the host striatal parenchyma. Immunohistochemical labeling of DAT showed two distinct regions in the putamen containing clusters of DAT-immunoreactive (−ir) dopamine neurons (FIG. 2D). Microscopic imaging of the entire transplanted and non-transplanted putamen showed markedly increased DAT labeling in the transplanted putamen compared with the non-transplant side, indicating robust reinnervation of the transplanted putamen from the engrafted CM-iPSC-derived dopaminergic neurons.

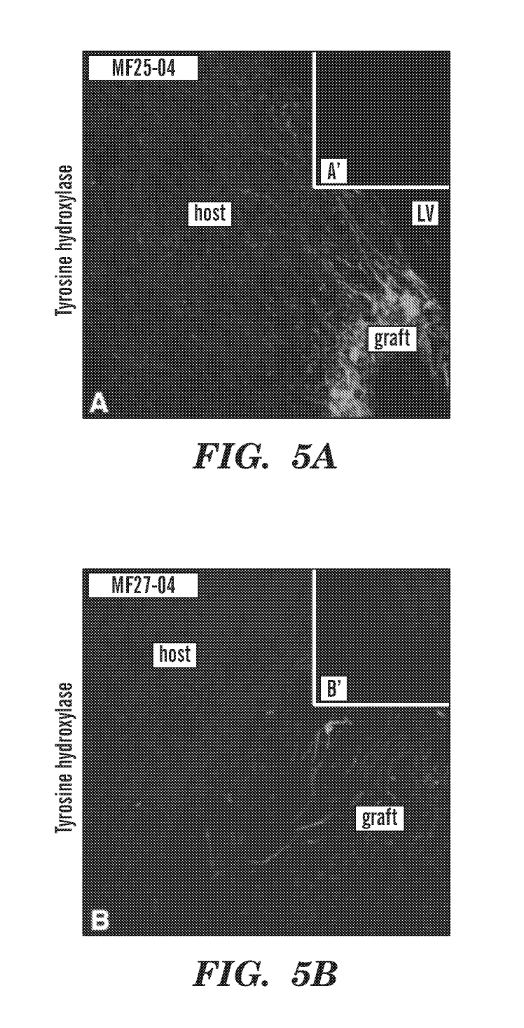

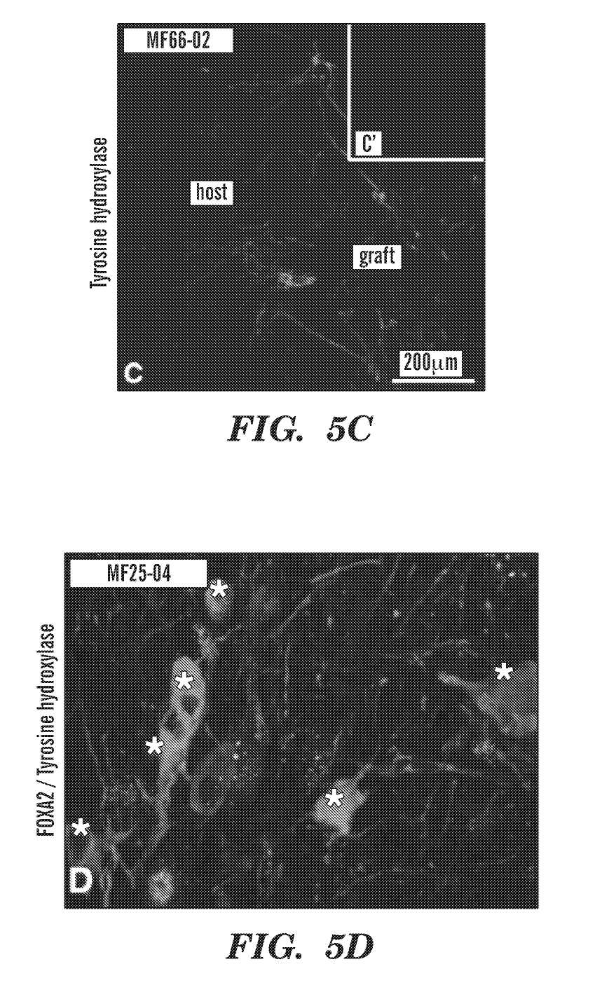

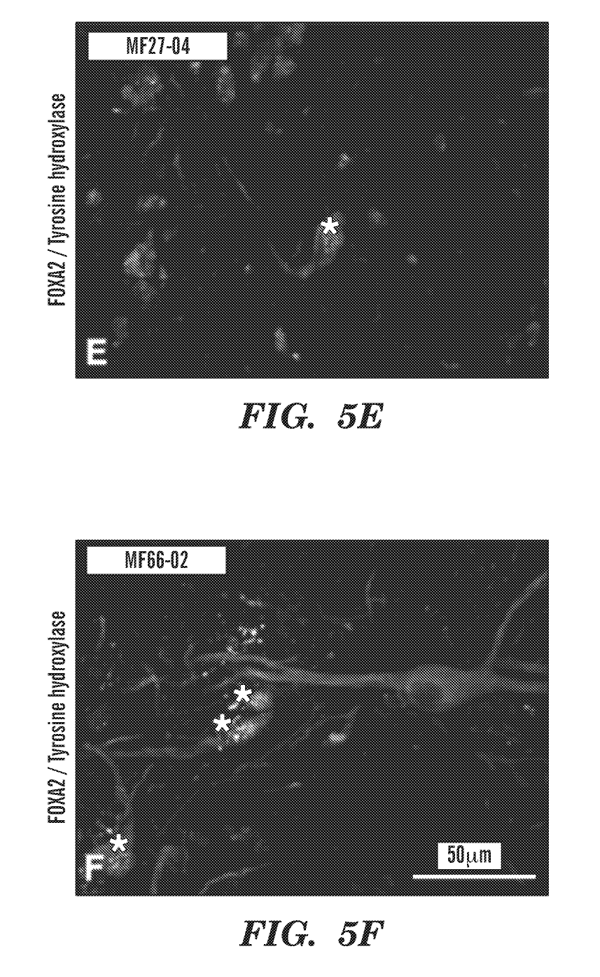

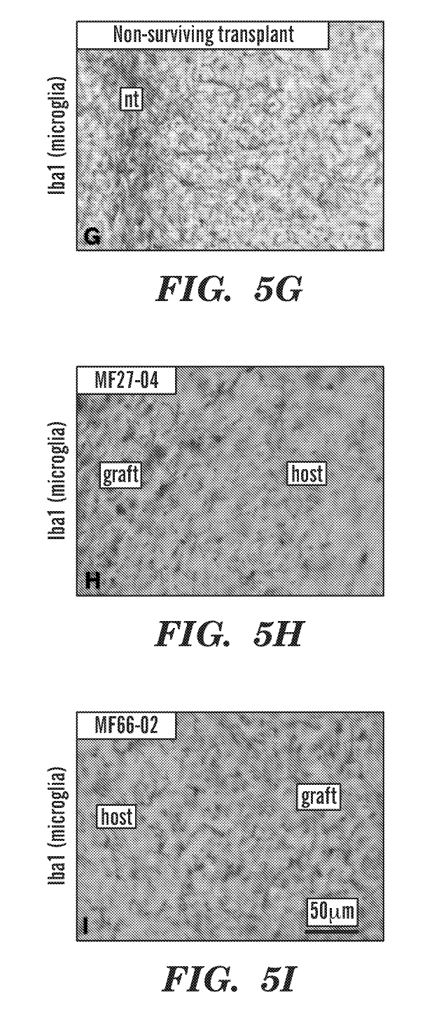

Stereological cell counts of TH-ir dopamine neurons showed the presence of 13,029 surviving transplanted dopaminergic neurons in the putamen. Engrafted TH-ir neurons (FIG. 2E) were located predominantly around the periphery of the grafts and extended axons into the host putamen, similar to the pattern of A9-like dopamine neurons in rodent and human fetal VM transplant cases (Mendez et al., 2005; Vinuela et al., 2008). As a comparison, the number of surviving TH-ir dopamine neurons in the transplanted putamen of the CMs MF27-04 (Sundberg et al., 2013) and MF66-02 was also determined. These cases received autologous transplantation of iPSC-derived dopamine neurons, but in contrast to MF25-04, did not exhibit any functional improvement at 1-2 years after transplantation. A total of 8,551 and 7,938 TH-ir dopamine neurons was present in the transplanted putamen of MF27-04 and MF66-02, respectively. A comparison of outgrowth of TH-ir fibers into the putamen from the grafts of MF25-04, MF27-04, and MF66-02 showed far more extensive dopaminergic reinnervation in MF25-04 (FIGS. 5A-5C), consistent with the functional recovery observed in this animal. These data indicate that improvement in motor function is achieved when an adequate number of transplanted midbrain dopamine neurons survive together with appropriate innervation of the transplanted putamen and that when there is not sufficient survival (less than 13,000 dopamine neurons) and poor dopamine axonal reinnervation of the putamen, CMs do not recover. These data also show that in this series of iPSC transplantations, the Cooper et al. (2010) neuron differentiation protocol was the most successful. Dopamine neuron transplantation works at an adaptive level to provide a sufficient number of appropriate A9 dopaminergic neurons (and their synapses) in the striatum to initiate movement (function). In accord with this, it has been previously shown following fetal dopamine neuronal transplantation in a rat unilateral PD model (Brownell et al., 1998b) that behavioral recovery only occurs after a threshold of new striatal dopaminergic synapses is reached (75%-85% of the intact striatum). With the development of iPSC-derived midbrain-like dopamine neurons for clinical use in PD, it will be essential to take into consideration that survival of a minimal required dose of transplanted midbrain-like dopamine neurons (Grealish et al., 2014) and sufficient reinnervation of the denervated putamen is necessary for improvement of PD motor symptoms.

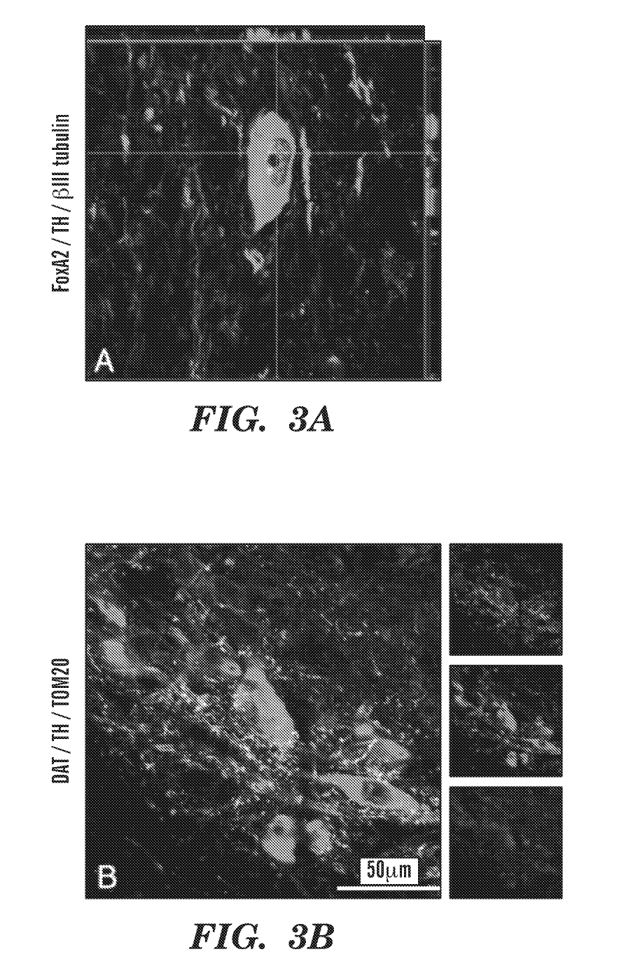

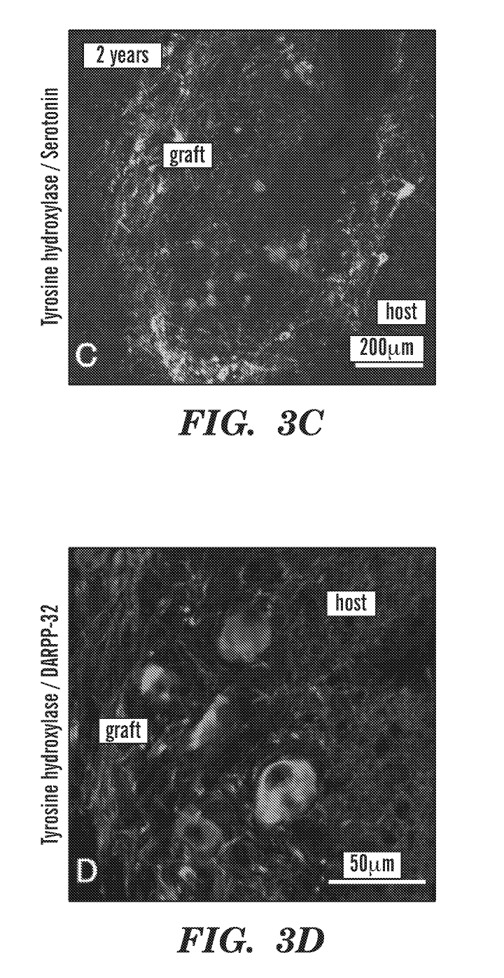

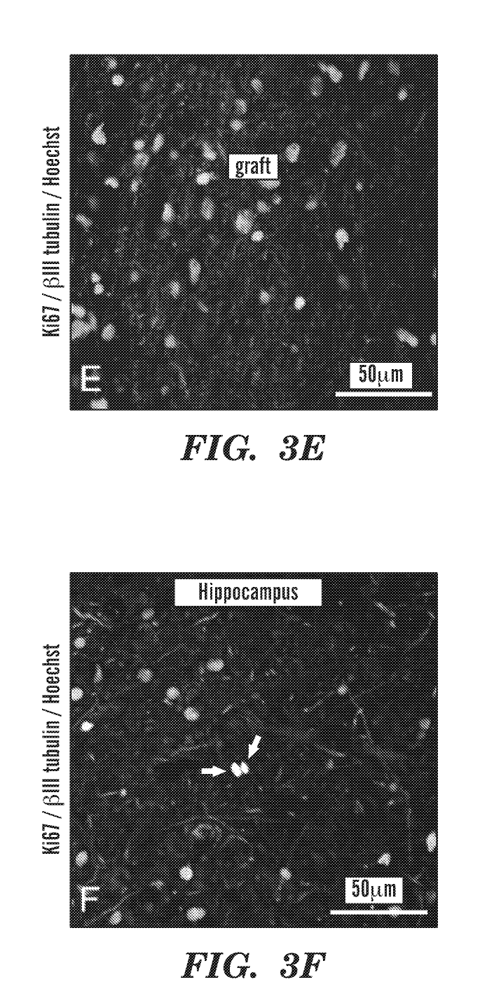

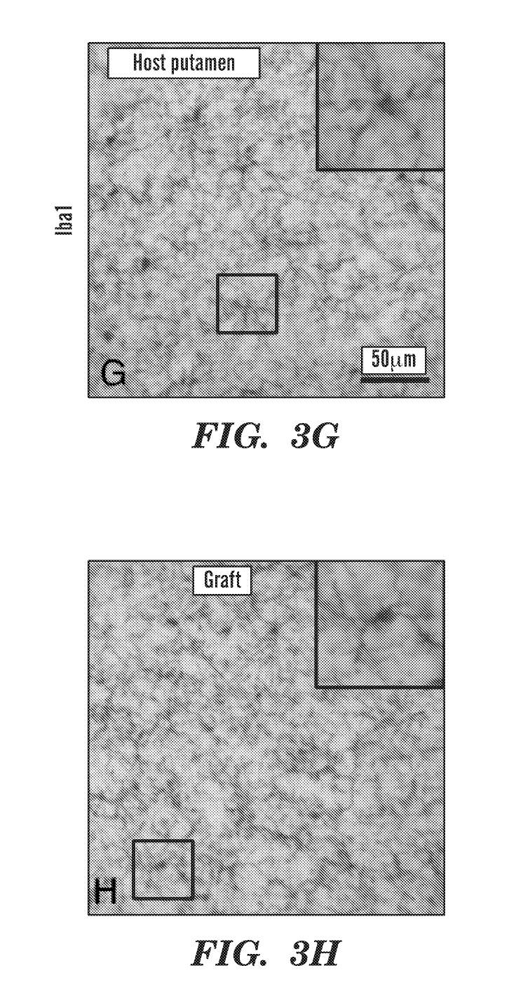

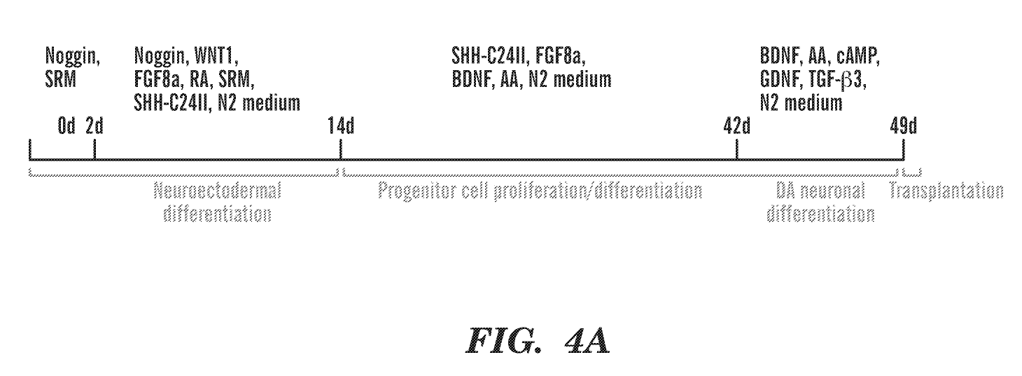

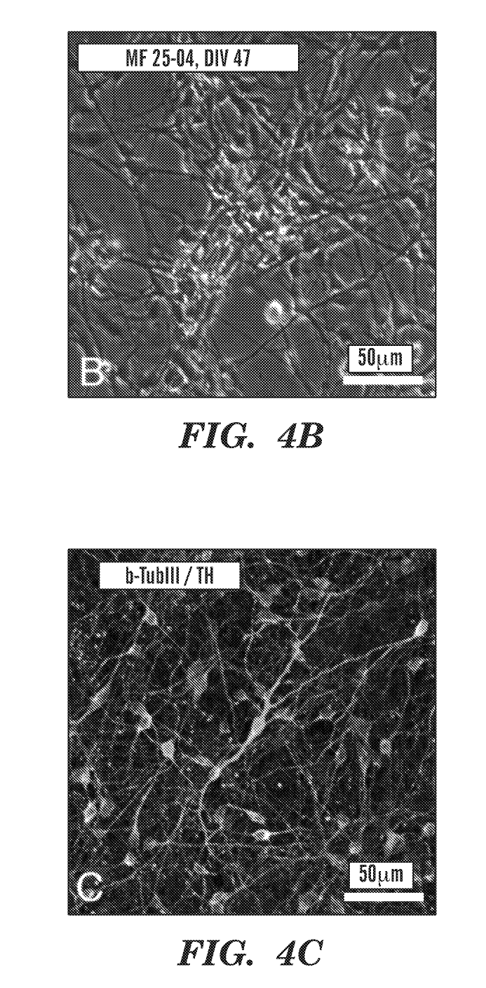

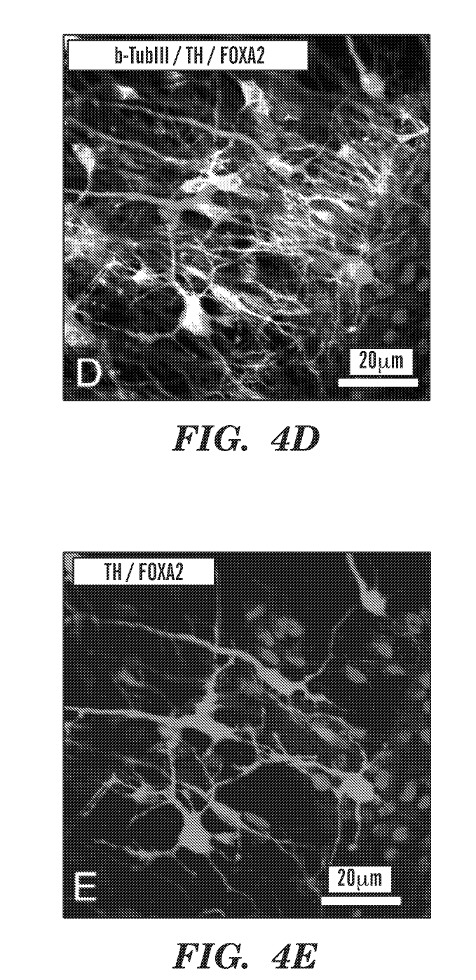

Colocalization of FOXA2/TH/bIII tubulin labeling confirmed the presence of midbrain-like dopamine neurons in the graft of MF25-04 (FIG. 3A). A separate FOXA2/TH labeling was performed in parallel in each of the three CM-iPSC grafts (MF25-04, MF27-04, and MF66-02) and showed more frequent FOXA2/TH colabeled (midbrain-like) dopamine neurons in the graft of MF25-04 compared with the grafts in MF27-04 and MF66-02 (FIGS. 5D-5F). A punctate expression of DAT along transplanted TH-ir cell bodies and processes confirmed the formation of mature synapses in MF25-04 (FIG. 3B). For an additional measure of neuronal health, the localization of mitochondria within TH-ir neurons was assessed using the mitochondrial marker, translocase of outer mitochondrial membrane 20 (TOM20) (Hallett et al., 2014). A homogenous localization of TOM20-ir mitochondria throughout the cell soma and processes of transplanted neurons in MF25-04 was observed (FIG. 3B), and there was no evidence of perinuclear accumulation or fragmentation of mitochondria, as has previously been reported during dopamine neuron stress or degeneration (Sterky et al., 2011).

Immunofluorescence labeling for 5HT (FIG. 3C) was performed to examine serotonergic neurons contained in the CMiPSC-derived transplant case, MF25-04. Serotonergic neurons were observed with a distribution and previously reported for rodent and human fetal VM grafts (Mendez et al., 2008; Vinuela et al., 2008). Labeling for striatal medium spiny GABAergic neurons in the grafted putamen using DARPP-32 (FIG. 3D) revealed robust DARPP-32 labeling in the host putamen perikarya and sparse DARPP-32-ir fibers at the graft-host border suggestive of some graft-host interaction as has been previously reported in fetal VM tissue (Doucet et al., 1989). Immunostaining with the proliferation marker, Ki-67, showed no positively labeled cells in the graft at 24 months after transplantation (FIGS. 3E and 3F). As a positive control, several Ki-67-labeled cells were observed in the dentate gyrus of the same animal (MF25-04). These data are in accord with the inventors' previous work using CM-iPSC-derived neural cell xenotransplantation in rats, where a low percentage of Ki-67-ir cells at 4 weeks after transplantation were observed, but no Ki-67-ir cells were found in mature grafts (16 weeks after transplantation) (Deleidi et al., 2011).

Histological analysis of microglial reactivity using immunohistochemistry for Iba1 revealed a local circumscribed increase in microglial cell density within the CM-iPSC-derived grafts (FIGS. 3G, 3H, 5H & 5I). However, Iba1 labeling was not different to that observed in the needle tracts of the ‘non-surviving” transplant group (FIG. 5G), and there was no immune reaction in the host putamen surrounding the autologous iPSC grafts. A prior report indicates that compared with autologous transplantation, allogeneic iPSC-derived neural cells elicit greater microglial activation, as well as MHCII (class II major histocompatibility complex) microglial expression and infiltration of leukocytes at ˜4 months after transplantation into the non-human primate brain (Morizane et al., 2013). The route of autologous transplantation, which requires no immunosuppression, is therefore a more successful route for cell therapy than using cell sources that are not completely matched to the donor. This work demonstrates in the successful case that dopamine synapse innervation is accompanied by the anticipated neurological improvement in parkinsonism. However, ˜13,000 primate midbrain dopamine neurons were needed to reach the threshold of functional improvements. Further optimization of this work can include the generation of consistent protocols to provide equivalent or greater midbrain dopamine neuron survival, for example, by increasing the dose of midbrain dopamine neurons, and scale up toward wide-scale use e.g., in clinical trials.

The present findings provide the first proof of concept preclinical study to demonstrate that autologous iPSC-derived midbrain-like dopamine neurons can engraft and survive over an extended period of time (at least 2 years), and improve motor function, in a non-human primate model of PD. Such autologous iPSC-derived dopamine neurons can provide remarkable and complete reinnervation of the denervated putamen without any immunosuppression. The inventors observed the most extensive dopaminergic axonal outgrowth reported in any study to date, including the inventors' previous studies and studies by others, after engraftment with iPSC-derived dopamine neurons in vivo (Emborg et al., 2013; Kikuchi et al., 2011; Sundberg et al., 2013). In addition, in the current study, no graft overgrowth, tumor formation, or inflammatory reaction was observed, which is consistent with previous rodent studies using iPSC-derived neural cells (Hargus et al., 2010; Sundberg et al., 2013; Wernig et al., 2008).

A general concern about the use of autologous transplantation is whether underlying PD-associated genetic mutations present in transplanted neurons would increase the vulnerability of midbrain dopaminergic neurons to disease pathology. However, such vulnerabilities do not preclude cell function at a fairly optimal level for at least 50-60 years from birth in a typical patient with such genetic risk (which represents a minority of PD cases). In addition, given how transplanted fetal neurons remain healthy for many years in PD patients, despite ongoing disease processes in the host brain (Hallett et al., 2014) and without wishing to be bound by theory, it is predicted that even in cases with severe genetic risk, iPSC-derived neurons are a reasonable strategy.

In summary, the present study clearly shows proof of concept for transplantation using iPSC-derived dopamine neurons in a preclinical context, and it is reasonable to infer that iPSC transplantation would provide clinical benefit and expected graft survival times similar to that observed with fetal transplantation (Barker et al., 2013; Hallett et al., 2014; Kefalopoulou et al., 2014). The data showing neurologically relevant functional improvements with concomitant positive neuroimaging are essential for the continued development and clinical translation of cell therapy using iPSCs. Overall, there is a strong immunological, functional, and biological rationale for using midbrain dopamine neurons derived from iPSCs for cell replacement in PD. Optimization of the methods can be performed to determine the optimal and safest dopamine neuron differentiation protocol to use, to evaluate the generation of different (non-neuronal) cell types, and to continue to refine the clinical protocols for generation of iPSCderived midbrain dopamine neurons to be used for transplantation to PD patients.

### REFERENCES

Barker, R. A., Barrett, J., Mason, S. L., and Bjorklund, A. (2013). Fetal dopaminergic transplantation trials and the future of neural grafting in Parkinson's disease. Lancet Neurol. 12, 84-91.
Brownell, A.-L., Jenkins, B. G., Elmaleh, D. R., Deacon, T. W., Spealman, R. D., and Isacson, O. (1998a). Combined PET/MRS brain studies show dynamic and long-term physiological changes in a primate model of Parkinson disease. Nat. Med. 4, 1308-1312.
Brownell, A. L., Livni, E., Galpern, W., and Isacson, O. (1998b). In vivo PET imaging in rat of dopamine terminals reveals functional neural transplants. Ann. Neurol. 43, 387-390.
Cooper, O., Hargus, G., Deleidi, M., Blak, A., Osborn, T., Marlow, E., Lee, K., Levy, A., Perez-Tones, E., Yow, A., and Isacson, O. (2010). Differentiation of human ES and Parkinson's disease iPS cells into ventral midbrain dopaminergic neurons requires a high activity form of SHH FGF8a and specific regionalization by retinoic acid. Mol. Cell. Neurosci. 45, 258-266.
Deleidi, M., Hargus, G., Hallett, P., Osborn, T., and Isacson, O. (2011). Development of histocompatible primate-induced pluripotent stem cells for neural transplantation. Stem Cells 29, 1052-1063.
Doucet, G., Murata, Y., Brundin, P., Bosler, O., Mons, N., Geffard, M., Ouimet, C. C., and Bjorklund, A. (1989). Host afferents into intrastriatal transplants of fetal ventral mesencephalon. Exp. Neurol. 106, 1-19.
Duan, W. M., Widner, H., and Brundin, P. (1995). Temporal pattern of host responses against intrastriatal grafts of syngeneic, allogeneic or xenogeneic embryonic neuronal tissue in rats. Exp. Brain Res. 104, 227-242.
Emborg, M. E., Liu, Y., Xi, J., Zhang, X., Yin, Y., Lu, J., Joers, V., Swanson, C., Holden, J. E., and Zhang, S. C. (2013). Induced pluripotent stem cell-derived neural cells survive and mature in the nonhuman primate brain. Cell Rep. 3, 646-650.

### EXEMPLARY METHODS AND MATERIALS

Generation of CM-iPSCs and Differentiation into Midbrain-Like Dopamine Neurons

Cynomolgus monkey (CM)-iPSCs were generated as previously described from 3 adult male Mauritian CMs, MF25-04, MF66-02 and MF27-04 (Deleidi et al., 2011; Sundberg et al., 2013). CM-iPSCs from MF25-04 were differentiated toward midbrain-like dopamine neurons over 49 days in vitro, and from MF66-02 and MF27-04 over 30 days in vitro, using an established differentiation protocols that support floor plate regionalization of cells into FOXA2 dopamine neurons (Cooper et al., 2010; Sundberg et al., 2013). The normal, developmental regional specialization of stem cells in vitro is based on the differentiation factors added to the cultures, and similar proportions of cell phenotypes are expected irrespective of the differentiation protocol used (e.g., Cooper vs. Sundberg). Moreover, the approximate percentage of dopaminergic neurons generated in the differentiation protocols (5-10%) is similar to the normal percent content of dopaminergic neurons in the developing midbrain (Isacson et al., 1995). Using the Cooper et al., 2010 differentiation of CM line MF25-04, at day 47, 2 days prior to transplantation, 51% of the cells were BIII-tubulin-immunoreactive (−ir) neurons, and 7% were tyrosine hydroxylase (TH)-ir dopaminergic neurons. 2% of the cells were labeled for TH and FOXA2 (a marker of the embryonic brain structure, the floor plate). Differentiated cultures stained negative for Oct4 indicating the absence of residual undifferentiated iPSCs. Using the Sundberg et al., 2013 differentiation of CM lines MF27-04 and MF66-02, in vitro pre-transplantation data showed that at day 28, 2 days prior to transplantation, 21-23% of the cells were BIII-tubulin-ir neurons, and 9% were TH-ir dopaminergic neurons. 1-2% of the cells were labeled for TH and FOXA2. Cell viability prior to transplantation was calculated using dye exclusion staining using 0.4% trypan blue solution (Sigma-Aldrich®). The number of unlabeled (live) cells (counted manually using a hemocytometer) were expressed as a percentage of the total (dead+live) cells.

MPTP Non-Human Primate Model of Bilateral PD and Behavioral Analyses

Induction of bilateral parkinsonism in CMs (n=10) using a systemic injection of MPTP was performed as previously described (Astradsson et al., 2009; Brownell et al., 1998; Hantraye et al., 1992; Wüllner et al., 1994). Animals were housed in individual home cages at the New England Primate Research Center (NEPRC). All studies were approved by the Harvard Medical School Institutional Animal Care and Use Committee (IACUC).

In brief, animals received intravenous administration of low doses of MPTP (Sigma-Aldrich®) diluted in normal saline every 1-2 weeks at 0.15-0.3 mg/kg per dose. Duration and dosage of MPTP administration was tailored for individual animals until stable, bilateral parkinsonism was achieved (Brownell et al., 1998; Jenkins et al., 2004; Sanchez-Pernaute et al., 2005). MPTP was administered for an average of 10±3 weeks (range 3-28 weeks), and the average cumulative MPTP dosage was 3.42±0.9 mg/kg (range 0.9-8.55 mg/kg). Parkinsonian motor symptoms were rated (weekly during MPTP administration and monthly after stable parkinsonism was established) using a parkinsonian rating scale (PRS) modified from the motor subscale of the Unified Parkinson's Disease Rating Scale (UPDRS) and developed for macaques (Imbert et al., 2000). The parkinsonian rating scale ranged from 0-24, with 24 being most severe. Global motor activity scores were collected using activity monitors (AW64 Actiwatch, Philips Electronics) (Mann et al., 2005) worn one week at a time every 2-4 months, and fine motor skills were assessed using a computerized reaching movement analysis panel (MAP) task (Gash et al., 1999). Cell transplantation paradigms (see below) were initiated within 2-4 years of reaching a stable parkinsonian baseline.

Statistical Analysis of Motor Behavior

Statistical analysis was performed using GraphPad Prism (Version 4.0) (GraphPad Software, Inc). For analysis of left or right fine motor skills over time (pre-transplantation, 12 months post-transplantation and 24 months post-transplantation) using the MAP task, a 1 way ANOVA followed by Tukey's Multiple Comparison test was performed. All tests were considered significant at p<0.05.

Neuroimaging for Dopamine Transporter (DAT) Function and Anatomical MRI.

Dopamine reuptake in the putamen was measured as previously described (Brownell et al., 1998) by PET studies and binding of the DAT tracer ¹¹C-CFT, prior to MPTP administration, at least 3 months after the last MPTP administration at the stable parkinsonian stage, and in MF25-04, at 2 Years Post-Transplantation of CM-iPSC-Derived Neural Cells.

Prior to transplantation of either CM-iPSC or Cyno-ES derived cells, each animal underwent magnetic resonance imaging (MRI) for structural imaging of the brain. Stereotaxic coordinates for transplantation were defined using e-film version 1.8.3 (Merge eFilm, Milwaukee) on MRI T2 weighted coronal and T1 weighted axial images with 3D reconstructions of the monkey brain obtained with the animal placed in the same stereotactic frame used for the surgery.

Transplantation of CM-iPSC-Derived Neural Cells

Four injection sites in the postcommisural putamen were defined by the increasing posterior distance (in millimeters) from the anterior commissure (AC) as: antero-posterior AC-1, AC-3, AC-5 and AC-7. Surgery was performed under sterile conditions. Anesthesia was induced with ketamine and maintained with isoflurane anesthesia with intubation. After cranial skin preparation, a midline skin incision was made in order to allow exposure of the target area and the muscle and fascia were thus retracted to expose the cranial surface and a burr-hole was drilled over the target area. The cell suspension was slowly (2 μl/min) injected along 4 mm at each anteroposterior site, using a blunt tip 20G Hamilton needle, with a wait time of 8 minutes at the conclusion of the injection in each tract. A total of 10 million neural cells were transplanted into the right post-commissural putamen of MF25-04, evenly distributed into 4 injection tracts at a density of 100,000 cells/μl. A total of 40 million neural cells differentiated from MF66-02 and MF27-04 CM iPSCs, were transplanted into the right (MF66-02) or left (MF27-04) post-commissural putamen, evenly distributed into 4 injection tracts and at a density of 200,000 cells/W.

After completion of the injections, the surgical site was washed, the burrhole was sealed with bone wax, and the fascia, muscle and skin were sutured in planes. The animals received cephazolin (20 mg/kg per 12 hour i.m.) and dexamethasone (1 mg/day i.m.) for 5 days and analgesia (buprenorphine 0.005 mg/kg per 12 hour i.m.) for 3 days. No cyclosporine was given. Corticosteroids were given postoperatively for 3 days as common neurosurgical practice to reduce edema and inflammation. The animals showed no complications and were followed for up to 2 years after transplantation. No animals in this study received any immunosuppression for the duration of the study.

Control Transplanted Animals

Three parkinsonian CMs received intra-putaminal allogeneic transplantation of 2-6 million differentiated parthenogenetic ES cells (Cyno-1), as previously described (Sanchez-Pernaute et al., 2005). Animals were followed for 1 year post-transplantation prior to post-mortem histological analyses. Analysis of graft survival showed that the grafts in each animal did not pass our criteria of at least 50 surviving dopamine neurons, and this series of animals were henceforth defined as the “non-surviving transplant” group.

Non-Transplant Control Animals

Four MPTP-lesioned parkinsonian CMs were, in parallel, used in this study as non-transplant control animals. These animals were followed for analysis of motor behavior and parkinsonism as described above for 2 years after induction of stable parkinsonism.

Immunocytochemistry and Immunohistochemistry

For pre-transplantation analysis of dopamine neurons, cultured cells were fixed in 4% paraformaldehyde and incubated in 10% normal goat or donkey serum for 1 hr at room temperature. Primary antibodies were added for 3 hrs at room temperature followed by incubation with fluorescent dye-conjugated secondary antibodies (Alexa Fluor goat or donkey anti-rabbit/mouse/sheep/goat 488/568/660; 1:500 [Molecular Probes, Eugene, Oreg.]) for 1 hr. Cultured cells from MF25-04 were analyzed at DIV47, 2 days prior to transplantation. Cultured cells from MF27-04 and MF66-02 were analyzed at DIV 28, 2 days prior to transplantation.

After completion of behavioral evaluations, animals were sedated with ketamine (15 mg/kg) and anesthetized with pentobarbital (Nembutal; 25 mg/kg, i.v.), and perfused transcardially with ice-cold heparinized saline followed by 4% paraformaldehyde. The brain was post-fixed for 18 hours, equilibrated in graded sucrose solutions (20%-30% in PBS) and sectioned on a freezing microtome in 40 μm slices that were serially collected. Sections were stained using either immunoperoxidase or immunofluorescence techniques. Sections for immunoperoxidase staining were treated for 30 min in 3% hydrogen peroxide (Humco, Texarkana, Tex., USA), washed 3 times in PBS, and incubated in 10% normal donkey serum (Vector Laboratories, Burlingame, Calif., USA) and 0.1% Triton X-100 in PBS for 60 min prior to 48 hr incubation at 4° C. with the primary antibody. After three 10-min rinses in PBS, the sections were incubated in biotinylated secondary antibody (donkey anti-rabbit/mouse/rat/sheep; Vector Laboratories, 1:300) in PBS at room temperature for 60 min. The sections were rinsed three times in PBS and incubated in streptavidin-biotin complex (Vectastain ABC Elite Kit; Vector Laboratories) in PBS for 60 min at room temperature. Following thorough rinsing with PBS, staining was visualized by incubation in 3,3′-diaminobenzidine solution (Vector Laboratories). For immunofluorescence, sections were rinsed for three times 10 min in PBS, incubated in 10% normal donkey serum (Vector Laboratories) and 0.1% Triton X 100 in PBS for 60 min and then incubated for 48 hours at room temperature in primary antibody. After an additional three 10 min rinses in PBS the sections were incubated in secondary antibodies (fluorescent dye-conjugated Alexa Fluor® donkey anti-rabbit/mouse/sheep 488/568/660; 1:500 [Molecular Probes]; Alexa Fluor® 488-conjugated streptavidin; 1:500 [Molecular Probes]; biotinylated goat anti-rat [Vector Laboratories]) in PBS with 10% normal donkey serum for 60 min at room temperature. Immunofluorescence staining was examined using a confocal microscope (LSM510 Meta; Carl Zeiss, Thornwood, N.Y., USA), and co-localization was confirmed by z-axis analysis. On selected sections, the primary antibody was omitted to verify specific staining.

The following primary antibodies were used: rabbit or sheep anti-TH, 1:300 [Pel-Freez]; rat DAT, 1:200 [Chemicon]; mouse anti-BIII tubulin, 1:1000 [Covance]; goat anti-FOXA2, 1:100 [Santa Cruz Biotechnology]; mouse anti-Oct4, 3 μg/mL [DSHB Iowa]; rabbit anti-5HT, 1:20,000 [ImmunoStar]; rabbit anti-Ki-67, 1:500 [Vector Labs], rabbit anti-DARPP-32, 1:500 [Cell Signaling], rabbit anti-Iba1, 1:300 [Wako].

Stereology of Grafted Dopamine Neurons

Stereological counts of grafted TH-ir neurons in MF25-04, MF66-02 and MF27-04 were performed using an unbiased, stereological cell counting method as previously described (Redmond et al., 2008). The optical dissector system consisted of a computer-assisted image analysis system including an Olympus BX-60 microscope hard-coupled to a Ludl computer-controlled x-y-z motorized stage, a high-sensitivity Hitachi 3CCD video camera system (Hitachi, Japan) and PC computer. Counts of TH-ir neurons were performed using Microbrightfield stereological software and calculated using an optical fractionator probe. The coefficient of error was used to assess probe accuracy and p<0.05 was considered acceptable.

### SUPPLEMENTAL REFERENCES

Astradsson, A., Jenkins, B. G., Choi, J. K., Hallett, P. J., Levesque, M. A., McDowell, J. S.,
Brownell, A. L., Spealman, R. D., and Isacson, O. (2009). The blood-brain barrier is intact after levodopa-induced dyskinesias in parkinsonian primates—evidence from in vivo neuroimaging studies. Neurobiol. Dis. 35, 348-351.
Brownell, A. L., Jenkins, B. G., Elmaleh, D. R., Deacon, T. W., Spealman, R. D., and Isacson, O. (1998). Combined PET/MRS brain studies show dynamic and long-term physiological changes in a primate model of Parkinson disease. Nature Med. 4, 1308-1312.
Cooper, O., Hargus, G., Deleidi, M., Blak, A., Osborn, T., Marlow, E., Lee, K., Levy, A.,
Perez-Tones, E., Yow, A., et al. (2010). Differentiation of human ES and Parkinson's disease iPS cells into ventral midbrain dopaminergic neurons requires a high activity form of SHH FGF8a and specific regionalization by retinoic acid. Mol. Cell Neurosci. 45, 258-266.
Deleidi, M., Hargus, G., Hallett, P., Osborn, T., and Isacson, O. (2011). Development of histocompatible primate-induced pluripotent stem cells for neural transplantation. StemCells 29, 1052-1063.
Gash, D. M., Zhang, A., Umberger, G., Mahood, K., Smith, M., Smith, C., and Gerhardt, G. A. (1999). An automated movement assessment panel for upper limb motor functions in rhesus monkeys and humans. J Neurosci. Methods 89, 111-117.
Hantraye, P., Brownell, A. L., Elmaleh, D., Spealman, R. D., Wullner, U., Brownell, G. L., Madras, B. K., and Isacson, O. (1992). Dopamine fiber detection by [11C]-CFT and PETin a primate model of parkinsonism. Neuroreport 3, 265-268.
Imbert, C., Bezard, E., Guitraud, S., Boraud, T., and Gross, C. E. (2000). Comparison of eight clinical rating scales used for the assessment of MPTP-induced parkinsonism in the Macaque monkey. J. Neurosci. Methods 96, 71-76.
Isacson, O., Deacon, T. W., Pakzaban, P., Galpern, W. R., Dinsmore, J., and Burns, L. H. (1995). Transplanted xenogeneic neural cells in neurodegenerative disease models exhibit remarkable axonal target specificity and distinct growth patterns of glial and axonal fibres. Nature Med. 1, 1189-1194.
Jenkins, B. G., Sanchez-Pernaute, R., Brownell, A. L., Chen, Y. C., and Isacson, O. (2004). Mapping dopamine function in primates using pharmacologic magnetic resonanceimaging. J Neurosci. 24, 9553-9560.
Mann, T. M., Williams, K. E., Pearce, P. C., and Scott, E. A. (2005). A novel method for activity monitoring in small non-human primates. Laboratory animals 39, 169-177.

### Example 2: Human iPSC Based Cell Therapy for Parkinson's Disease

### Exemplary Clinical Product and Use Thereof

In one embodiment, the transplantation composition is GMP grade, Parkinson's disease (PD) patient-derived foot-print-free induced pluripotent stem cell (iPSC), differentiated toward a midbrain dopaminergic fate, quality-checked, frozen and thawed.

PD patient iPSCs are derived from peripheral blood mononuclear cells (PBMCs) using an episomal iPSC reprogramming Kit. The reprogramming kit contains 5 episomal (non-integrating) vectors, Oct4, Sox2, Lin28, Klf4, and L-Myc (Okita K et al., Nature Methods 8, 409-412, 2011) and produces transgene-free, virus-free iPSCs. The episomal vectors are introduced into the cell by electroporation. The iPSCs are produced and grown in a GMP facility and quality checked to have a normal karyotype, ability to form teratoma, expressing stem cell (pluripotency) markers and be free of endotoxin and microorganisms.

Differentiation into dopaminergic (DA) neurons from iPSCs occurs under xeno-free conditions in a GMP facility using small molecules that promote ventral midbrain (VM) dopamine neuron specification. The resulting cell product is a VM-like cell preparation that contains VM dopamine neurons at levels similar to the fetal ventral midbrain. The cell preparation is quality checked to be free of microorganisms and endotoxin, express the correct cell-surface markers and be devoid of pluripotent stem cells. In one embodiment, the cells are depleted of TRA160- and SSEA-expressing cells by removing cells that bind to antibodies for TRA160 and/or SSEA. The quality checked batches of cells are stored frozen until use. Each GMP-grade cell preparation is thawed and tested thoroughly in vitro and in in vivo bioassays for safety prior to clinical use.

In one embodiment, autologous iPSC-derived midbrain dopamine neurons are delivered by stereotactic intraputaminal injection in patients having or suspected of having Parkinson's disease. In one embodiment, transplantation is unilateral or bilateral and into the postcommissural putamen. Typically, several (e.g., 2, 3, 4, 5, or more) putaminal targets are identified or calculated from the patient's MRI scan.

Typically, an appropriate cell dose of 20-50 million cells in 160-320 μl volume is injected over 4 injection tracts in the putamen. In one embodiment, the transplantation procedure comprises stereotactic injection, using a syringe needle connected to an injector system, using frame-based or frameless navigation. It is important to note that when autologous cells derived from the patient's own somatic cells are used, no immune suppression is needed or required following the transplantation. Efficacy of the transplantation can be determined using clinical assessments routinely used for PD patients (e.g., UPDRS), and can optionally comprise additional electronic devices to obtain activity and dyskinesia, and serial PET scans to establish presence of surviving implanted dopamine neurons and have an in vivo readout of changes over time.

### Conversion of the Differentiation Protocol to Xeno-Free Conditions for a Cell Product Safe for Human Transplantations

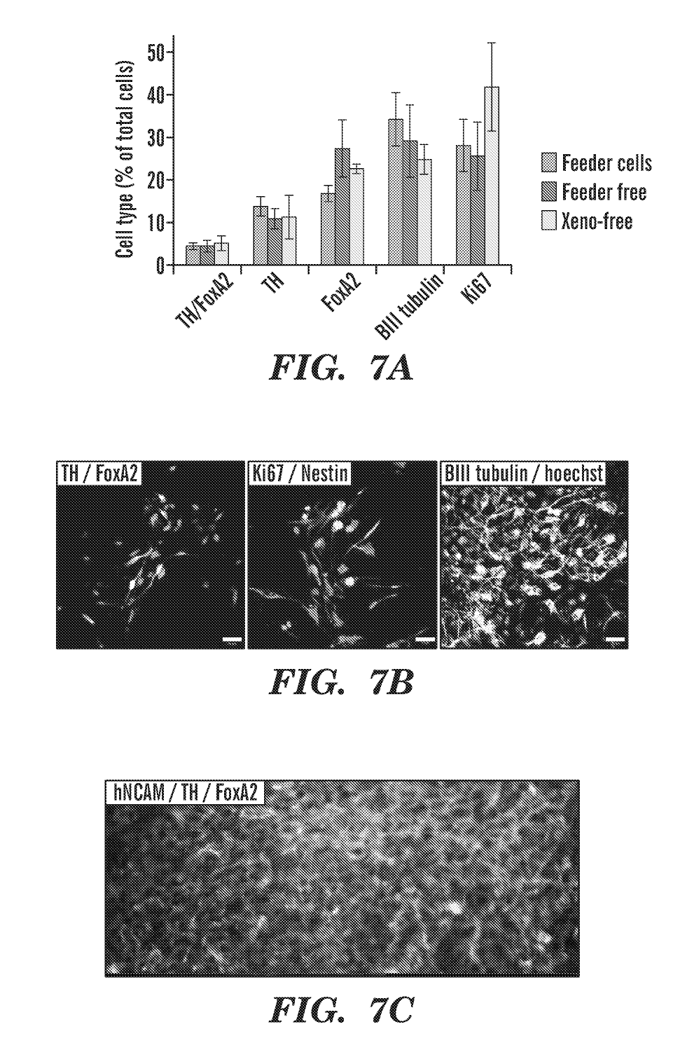

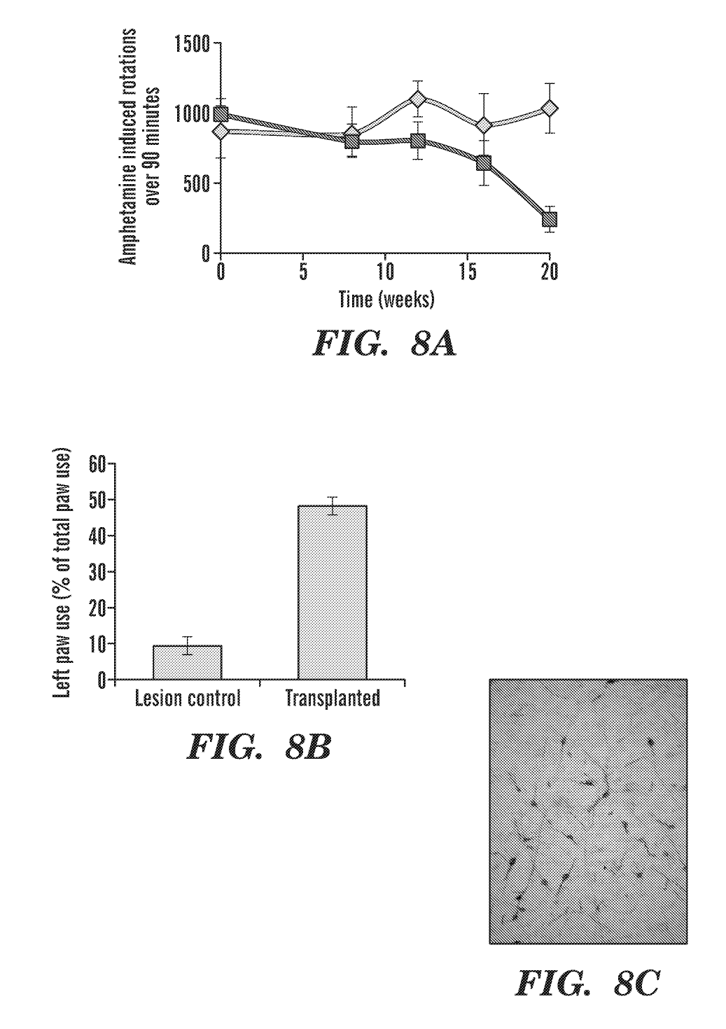

The basic differentiation protocol previously published by the inventors (Cooper et al., 2010) that differentiated iPSCs into midbrain DA neurons contained components incompatible with human transplantations, such as initiation of the protocol on mouse feeder cells and use of non-human recombinant protein and media sources. Thus, the inventors developed a xeno-free version of the previous protocol, resulting in cell products of comparable composition and quality. In this protocol the mouse feeder cells are replaced with a humanized substrate, the recombinant proteins are replaced by small molecules and all of the media products used are xeno-free media products. The percentage of midbrain DA neurons is equivalent to what was observed with the Cooper et al. protocol and within the range of a normal fetal midbrain. When transplanted into immunosuppressed rat striatum the cells differentiated with the modified protocol survive (FIG. 7). The cells also survive long-term in immunosuppressed rats and improve the motor asymmetry of rats with DA neuron loss (FIG. 8).

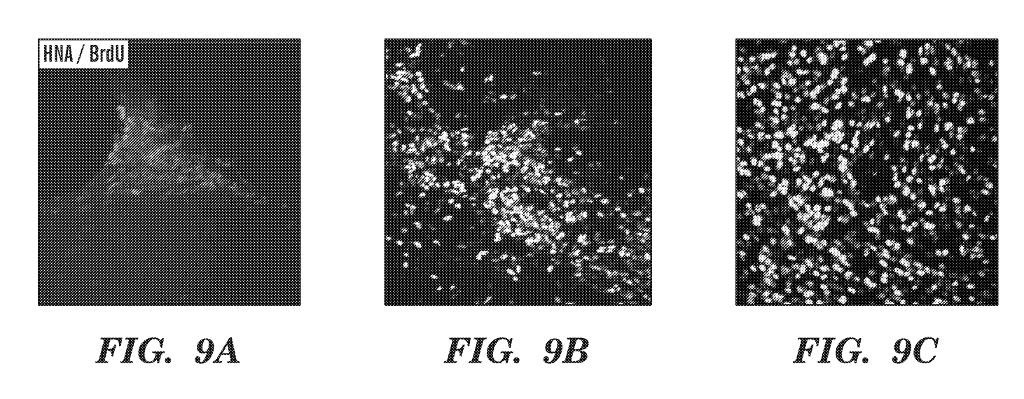

Although there are proliferating cells present in the cell preparation prior to transplantation, these cells appear to terminally differentiate and mature in the graft with time. Pre-transplantation in vitro data indicate that a majority of these proliferating cells are positive for the intermediate filament protein nestin, a marker observed in neural precursors and not pluripotent cells. In order to determine if proliferating cells are present in the graft, a BrdU study was performed on rats 7 weeks and 6 months after transplantation. Seven weeks after transplantation, some scattered BrdU positive cells were observed in rats given BrdU (50 mg/kg/day) for 12 days, indicating dividing cells. However, in the mature grafts, 6 months after transplantation, the inventors did not detect any graft-derived proliferating cells (FIG. 9). Additionally, tumor formation has not been observed in the rats after transplanting these cell preparations (n>300 rats over 6 years).

1. A composition for the treatment and/or prevention of Parkinson's Disease, the composition comprising:
(a) a population of cells comprising at least 3% dopaminergic neurons, wherein the dopaminergic neurons express FOXA2, β-tubulin, and tyrosine hydroxylase (TH),
(b) a neurotrophic factor, and
(c) a pharmaceutically acceptable carrier.

2. The composition of claim 1, wherein the neurotrophic factor is selected from the group consisting of: glial cell line-derived neurotrophic factor (GDNF), neurturin (NRTN), artemin (ARTN), and persephin (PSPN).

3.-4. (canceled)

5. The composition of claim 1, wherein the population of cells comprises between 4-40% dopaminergic neurons.

6. The composition of claim 1, wherein the population of cells is free of teratoma-forming cells.

7.-9. (canceled)

10. The composition of claim 1, wherein the volume of the composition is less than 1 mL.

11. The composition of claim 1, wherein the population of cells comprises at least 10,000 dopaminergic neurons.

12.-13. (canceled)

14. The composition of claim 1, wherein the dopaminergic neurons further express G-protein inwardly-rectifying potassium channel (GIRK)-2.

15. A method for treating and/or preventing Parkinson's disease, the method comprising: administering a composition of claim 1 into the caudate, putamen, nucleus accumbens, or subthalamic nucleus of a subject in need thereof, thereby treating and/or preventing Parkinson's disease.

16. The method of claim 15, wherein the composition is administered to the putamen.

17. The method of claim 15, wherein the composition is administered bilaterally.

18. The method of claim 15, wherein the composition is administered using stereotactic injection.

19.-20. (canceled)

21. The method of claim 15, wherein the subject is a human.

22.-23. (canceled)

24. The method of claim 15, wherein the method restores the at least 60% of the functional dopaminergic synapses normally observed in the putamen.

25. The method of claim 15, wherein the method reduces the dose of L-DOPA required by the subject to achieve adequate clinical control of symptoms.

26. A method for reducing the dose of L-DOPA required by a Parkinson's patient to achieve adequate clinical control of symptoms, the method comprising:
(a) administering a composition of claim 1 into the caudate, putamen, nucleus accumbens, or subthalamic nucleus of a subject receiving L-DOPA to control Parkinson's symptoms,
(b) monitoring the subject for engraftment of dopaminergic neurons and/or for side effects associated with L-DOPA, and
(c) reducing or eliminating the dose of L-DOPA as required to reduce L-DOPA associated side effects while maintaining adequate clinical control of Parkinson's symptoms.

27. (canceled)

28. A method comprising:
(a) reprogramming somatic cells obtained from a subject into iPS cells,
(b) contacting the iPS cells with retinoic acid, human SHH and FGF8A to promote differentiation of the iPS cells into dopaminergic neurons, wherein the dopaminergic neurons express FOXA2, β-tubulin and tyrosine hydroxylase, and
(c) depleting the population of cells of step (b) of teratoma-forming cells.

29. The method of claim 28, wherein the population of cells of step (c) comprises at least 4% dopaminergic neurons that express FOXA2, β-tubulin and tyrosine hydroxylase.

30. The method of claim 28, wherein the dopaminergic neurons further express GIRK2.

31. The method of claim 28, wherein the teratoma-forming cells are depleted from the population of cells of step (b) by removing cells expressing TRA160 and/or SSEA.

32. The method of claim 28, wherein the somatic cells are peripheral blood mononuclear cells (PBMCs) or fibroblasts.

33.-35. (canceled)
